(19) United States
(12) Patent Application Publication
(10) Pub. No.: US 2009/0220490 A1
(43) Pub. Date: Sep. 3, 2009

(54) Compositions and Assays for Inhibiting HCV Infection

(76) Inventor: Martina Buck, Del Mar, CA (US)

Correspondence Address: SUTHERLAND ASBILL & BRENNAN LLP, 999 PEACHTREE STREET, N.E., ATLANTA, GA, 30309, US

(21) Appl. No.: 12/280,367

(22) PCT Filed: Feb. 23, 2007
(86) PCT No.: PCT/US2007/062713

§ 371 Date: Feb. 27, 2009

Related U.S. Application Data
(60) Provisional application No. 60/776,119, filed on Feb. 23, 2006.

(51) Int. Cl. A61K 39/395; C07K 2/00; C07K 16/00; A61K 38/16; A61K 38/00; A61K 39/00; C12N 5/08; A61P 31/12

(52) U.S. Cl. 424/130.1; 530/300; 530/387.1; 514/2; 514/12; 424/184.1; 435/370

(57) ABSTRACT

The present invention provides isolated compounds, peptides, antibodies, vaccines that inhibit one or more functional domains of HCV E2 protein from interacting with associated proteins selected from the group consisting of AP-50, HSC70, Cyclin A, and Cyclin G. Pharmaceutical compositions and method of use thereof comprising the same for inhibiting HCV infection are also provided. The present invention further provides a primary hepatocyte cell culture comprising hepatocytes from a health individual and bodily fluid from a HCV infected individual, and method of use thereof, for screening compounds for inhibiting HCV infection.

### REFERENCE TO RELATED APPLICATIONS

This application claims a priority of U.S. Provisional Application Ser. No. 60/776,119, entitled “Assays and Treatments for Virus Infections and Other Disease Conditions,” filed Feb. 23, 2006, the entire application is incorporated by reference herewith.

### STATEMENT OF GOVERNMENT INTEREST

This invention was made with government support. As such, the U.S. Government may have certain rights in this invention.

### FIELD OF THE INVENTION

The present invention relates to HCV infection and HCV related diseases. In particular, the present invention relates to methods to culture primary hepatocytes, assays for screening compounds to inhibit HCV and/or other virus infections, and pharmaceutical compositions comprising peptides/lead compounds for preventing and treating HCV infections and related diseases.

### BACKGROUND OF THE INVENTION

HCV is a Hepacivisue, from the family Flaviviridae (B. D. Lindenbach et al., in Fields Virology, D. M. Knipe, P. M., Howley, eds. (Lippincott-Raven, Philadelphia, 2001), pp. 991-1041), which include three genera of small-enveloped positive-strand RNA viruses (B. Robertson et al., Arch, Virol. 143, 2493 (1998)). The HCV 9.6-kb genome consists of a single open reading frame (ORF) flanked by 5′ and 3′ nontranslated regions (NTRs) (K. J. Blight et al., in Hepatitis viruses, J. H. J. Ou, ed. (Kluwer Academic Publishers, Boston, 2002), pp 81-108). The HCV 5′ NTR contains an internal ribosome entry site (IRES), mediating cap-independent translation of the ORF of ˜3011 amino acids. The resulting polyprotein is processed into 10 proteins. Host signal peptidase cleavages within the N-terminal portion of the polyprotein generate the structural proteins core (C), E1 and E2 proteins.

The mechanisms of hepatitis C virus (HCV) cell entry, trafficking, viral assembly, and exit are poorly understood. E2 has been shown to dimerize with E1, and associate with the cellular CD 81 receptor (P. Pileri et al., Science 282, 938 (1998)) and the LDL receptor (S. Seipp et al., J Gen Virol 78, 2467 (1997)), although neither association has proven to be the exclusive cellular entry mechanism. The intracellular role of E2 remains unknown.

The hepatocyte is the primary target cell for HCV, although various lymphoid populations, especially B cells and dendritic cells, may also be infected at lower levels (Hayashi, J. Immunol. 162, 5584-5591 (1999); Auffermann-Gretzinger, Blood 97, 3171-3176 (2001)). HCV infection studies have also involved infected patients (Oldach et al., J. Exp. Med. 194, 1395-1406 (2001); Takaki et al, Nat. Med. 6, 578-582 (2000); Lechner et al, J Exp. Med. 191, 1499-1512 (2000)).

HCV is one of viruses that can not or can only poorly be propagated in cell culture and present the major challenge to culture this pathogen. Recently, Heller et al (Proc. Natl. Acad. Sci. USA 102, 2579 (2005)), Lindenbach et al (Science 1114016 (2005)), Zhong et al (Proc. Natl. Acad. Sci. USA 102, 9294 (2005)) and Wakita et al (Nat. Med. (2005)) were able to replicate genomic HCV in Huh-7-derived cells with the efficient production of HCV viral particles that were infectious to cultured Huh-7-derived cells. Logvinoff et al. (Proc. Natl. Acad. Sci. USA 101, 10149-10154 (2004)), Shoukry (J. Immunol. 172, 483-492 (2004)), Thimme (Proc. Natl. Acad. Sci. USA 99, 15661-15668 (2002)), and Bukh (Hepatology 39, 1469-1475 (2004) were also able to replicate genomic HCV in chimpanzees.

Therefore, at present the state of the art is/are systems that express one or two viral proteins in modified cells or animal models (Liver Int. 2005, Feb. 25(1):141-7; Gastroenterology. 2005, Feb, 128(2):334-42; Exp Mol Med. 2004, Dec. 36(6):588-93; and Hepatology, 2005 Feb. 41(2):265-74). However, these transfected and transgenic cells and/or animal models may not accurately or completely reflect mechanisms for HCV infection. For instance, Huh-7 hepatocellular carcinoma cell line may not be an accurate reflection of the vial protein mechanisms involved in HCV infection because these carcinoma cells have abnormal endocytic pathways (G. Kroemer et al., Nat Rev Cancer 5, 886 (2005)), and may not been possible to establish whether HCV endocytosis is the same in these tumor cell lines and in normal hepatocytes (Jones et al., Science 279, 573 (1998); Damm et al., J Cell Biol 168, 477 (2005)). Furthermore, neither proliferation, endocytosis, nuclear transportation, signaling, mitochondrial function, nor metabolism of carcinoma cells is normal. Moreover, only limited HCV genotype, e.g., genotype 2, from a single patient may be tested in these tumor cell lines, and no special patient populations can be tested in the carcinoma cell line. In addition, the chimpanzee models are costly and do not allow for large-scale screening. As a result, use of these hepatocellular carcinoma cell culture systems to test potential therapeutics for HCV could generate false positive or negatives that could result in the loss of a promising drug or investment in a weak drug.

Hepatitis C virus (HCV) induces an acute illness and, in over 50% of the infected individuals, will develop into chronic hepatitis. Infected individuals are also at risk of developing hepatocellular carcinoma (HCC) and/or cirrhosis. The global prevalence of chronic HCV is 3% of the population, with approximately 2 new cases per 100,000 persons annually. At present, the cellular mechanisms of HCV infection are not known, and there is no treatment that the majority of patients with HCV respond to. The current therapeutic approach for treating HCV is interferon or interferon plus ribavirin, which is currently the only treatment for HCV infection. These therapies have had, overall, positive effects (approximately a 50% response rate) but there are also serious side effects associated with these therapies. The current treatment also does not eradicate the virus.

Given these factors, there is a need to develop therapeutics for disrupting and blocking HCV infection via eradicating the virus. There is also a need to develop a normal hepatocyte cell culture system that is suitable for HCV infection and proliferation, and accurately and/or completely reflects the HCV life cycle and host-virus interactions after the HCV infection, so that such normal cell culture system, when infected with HCV, can be used as a tool for screening and developing therapeutics for HCV.

### SUMMARY OF THE INVENTION

The present invention provides an isolated compounds, peptides, antibodies, vaccine, preferably peptides, for inhibiting HCV infection comprising a peptide that inhibits one or more functional domains of HCV E2 protein from interacting with associated proteins selected from the group consisting of AP-50, HSC70, Cyclin A, and Cyclin G. In one preferred embodiment, the isolated peptide binds to an amino acid sequence of LIXXQXTG (SEQ ID NO: 1), SGREYALKR (SEQ ID NO:32), or LVGLLTPGAKQNIQLI (SEQ ID NO:33), of the HCV E2 envelop protein. In yet another preferred embodiment, the isolated peptide is an AP-50 mutant. In yet another preferred embodiment, the isolated peptide comprises an AP-50 mutant comprising an amino acid sequence of QGAVQ (SEQ ID NO:2) having an Alanine substitution at position 156 (Ala1156) of a native AP-50.

In yet another preferred embodiment, the isolated peptide comprises an AP-50 mutant that lacks a functional domain of a native AP-50. Preferably, the functional domain is a J domain of a native AP-50. In yet another preferred embodiment the isolated peptide is mutant of HCV associated proteins including, but not limited to HCV E2, HSC70, Cyclin C, and Cyclin C. In yet another preferred embodiment, the isolated peptide comprises a HCV E2 mutant having less PI-3K activating capacity than native HCV E2 protein.

The present invention also provides isolated nucleotides encoding the aforementioned proteins or peptides that are capable of interacting with HCV NS1/E2 envelop protein or AP-50 to disrupt and inhibit HCV infection.

The present invention further provides a pharmaceutical composition for preventing and/or treating HCV infection comprising the isolated compounds, preferably peptides, of the present invention, and one or more pharmaceutically acceptable carrier. The present invention further provides antibodies and vaccines generated from, and/or comprising the isolated peptides of the present invention for HCV prevention and/or treatment. Moreover, methods for preventing or treating HCV infection comprising administering to a subject at need an effective amount of pharmaceutical composition comprising the compounds, peptides, mutants, analogs, antibodies, vaccines thereof, of the present invention are also provided.

The present invention also provides a primary hepatocyte cell culture comprising hepatocytes derived from a healthy subject and a bodily fluid derived from a HCV infected subject. In one preferred embodiment, the bodily fluid is serum or plasma In yet another preferred embodiment, the primary hepatocyte cell culture of comprises HCV genotypes 1, 2, 3, 4, or combinations thereof. In yet another preferred embodiment, the subject is a human.

The present invention further provides a method for screening a compound for inhibiting HCV infection. Such method comprises a) obtaining the primary hepatocyte cell culture of claim 22, b) infecting said primary hepatocyte cell culture with HCV in the absence or presence of said compound, and c) determining differences of HCV infection in the cultures in the absence or presence of said compound.

### BRIEF DESCRIPTION OF THE DRAWINGS

FIG. 1 illustrates HCV Infection of the human hepatocyte culture system. Day-3, primary human hepatocytes were infected with HCV genotype 1 (inoculum: 11,200 HCV virions) as described in Methods. A) Confocal microscopy was performed for nuclei (TO-PRO-3; blue), HCV E2 (red) or HCV core (green). Twenty-four hours infected hepatocytes expressed HCV E2 and core proteins, while control hepatocytes had only background fluorescence. B) and C) Transmission electron microscopy was performed in human hepatocytes infected for 4 hr. HCV virions were detected in the perinuclear region (arrow). B: (×27,500) and C: (×55,000) panels.

FIG. 2 illustrates HCV amplification in the Human Hepatocyte Culture System. Day-3 primary human hepatocytes were infected with HCV genotype 1 (56,000 HCV virions); genotype 2 (68,000 HCV virions); genotype 3 (22,400 HCV virions); or genotype 4 (41,800 HCV virions) for up to week-3 as described in Methods. A) HCV virions in the media were purified by affinity chromatography. Immunoblotting for E2 and core proteins were done in HCV lysates at time zero (inoculum) (lane 1) and at 72 hr (lane 2). B) HCV RNA was quantified in primary human hepatocytes infected with HCV genotype 1 (closed bars); genotype 2 (open bars) and genotype 4 (hatched bars) as described in (A) at day-2, week-2 and week-3, and from livers of two HCV-infected patients. Results from quadruplicate samples of three independent experiments are shown. C) HCV E2 and core were detected by immunoblotting from human hepatocytes cell layers. E2 and core were expressed in HCV genotype 1-infected hepatocyte cultures for 24 hr (lane 2), day 3 (lane 3), day 6 (lane 4), day 10 (lane 5), day 13 (lane 6), day 15 (lane 7), and day 21 (lane 8), compared to time-zero HCV infection (lane 1). D) Day-3 primary human hepatocytes were infected with HCV genotypes 1, 2, 3 and 4 (lanes 1-4) for 24 hr as described in (A). HCV E2 was detected by immunoblot ing from human hepatocytes cell layers. Values are those of HCV-infected hepatocytes minus the background values of time-zero HCV-infected hepatocytes (˜10%). Results from triplicate samples of three independent experiments are shown. E)*HCV infection was quantified by immunopurification of HCV E2 in naïve primary human hepatocytes infected with HCV genotypes 1, 2, or 3 (open bars; lanes 4-6) produced by human hepatocytes infected with HCV genotypes 1, 3 or 4 for 72-hr (closed bars; lanes 1-3) as described in (A). Results from triplicate samples of two independent experiments are shown. F) [³⁵S]-methionine labeling of naïve primary human hepatocytes infected with HCV genotypes 1, 3 and 4 (lanes 1-3) as described in (E). HCV infection was quantified by determining the radioactivity of immunopurified HCV E2; background radioactivity was negligible.

FIG. 3 illustrates that HCV E2 associates with AP-50 in HCV-infected human liver and HCV-infected human hepatocyte cultures. A) AP-50, E2 and (3-actin immunoblots were performed on E2 immunoprecipitates from protein lysates obtained from HCV-infected livers (lanes 2 and 3). Uninfected liver immunoprecipitates were used as negative control (lane 1). HCV E2 and AP-50 were associated in HCV-infected livers. B) Confocal microscopy was performed for nuclei (TO-PRO-3; blue), HCV E2 (red) or AP-50 (green). HCV E2 and AP-50 co-localized in a HCV-infected human liver (merge). C) AP-50, E2 and β-actin immunoblots were performed on HCV E2 immunoprecipitates from protein lysates obtained from serum-derived HCV-infected human hepatocyte cultures, genotype 1 (lanes 2-4), genotype 3 (lanes 5-7), and genotype 4 (lanes 8-110). Immunoprecipitates from time zero-infected human hepatocytes were used as control (lane 1), as described in Methods. HCV E2 and AP-50 were associated in HCV-infected human hepatocytes. D) Confocal microscopy was performed for nuclei (TO-PRO-3; blue), HCV E2 (red) or AP-50 (green).

FIG. 4 illustrates that AP-50 and HCV E2 associate in HCV-infected human liver. E2, AP-50 and β-actin immunoblots were performed on reciprocal AP-50 immunoprecipitates from protein lysates obtained from HCV-infected livers (lanes 2 and 3). Uninfected liver immunoprecipitates were used as negative control (lane 1). 12 and AP-50 were associated in HCV-infected livers.

FIG. 5 illustrates that HCV E2 has a kinase catalytic loop and homology to the kinase domain of GAK. A. The consensus catalytic loop of CDKs compared to that of E2, with the mutation K25R (blue). B. The HCV E2 associates with mouse cyclin G on an immunoblot. (lanes 1. HCV E2 wt, 2. K25R, 3. L197A, 4. Y228E, 6. Y228F, 7. E271A, 8. L283A, 9. L292A, 10. I313A, 11. I331A, 12. L342A). Control Immunopurifications of untransfected cells had no E2 protein, (data not shown). C. The HCV E2 (green) co-localizes (yellow), with human cyclin A (red) by immunostaining. Nuclei are stained with TO-PRO3, (blue). D. The alignment of HCV E2 (green) with GAK (black) is shown with mutations (blue).

FIG. 6 illustrates that HCV E2 associates with Cyclin A in primary human hepatocytes. A. Cells transfected as described. Reciprocal immunopurifications of E2 and immunoblots of Cyclin G, Ap50, HSC 70, and E2 are shown. (lanes 1. HCV E2 wt, 2. K25R, 3. L197A, 4. Y228E, 5. Y228F, 6. E271 A, 7. D274A, 8. L283A, 9. L292A, 10. I313A, 11. I331A, 12. L342A). Control Immuno-purifications of untransfected cells had no E2 protein, (data not shown). B. The HCV E2 protein (green) transfected into primary human hepatocytes is shown to co-localize (yellow) with human cyclin A (red) by immunostaining. Nuclei are stained with TO-PRO3, (blue). C. Primary human hepatocytes transfected as above and Cyclin A immunopurified with immunoblots for Cyclin A and E2. E2 association with human Cyclin A is shown in lane 2. Lane 1 is a control without E2.

FIG. 7 illustrates that HCV E2 phosphorylates AP2 subunit AP50/μ2. A. AP50 immunopurification from above cells. (lanes 1. HCV E2 wt, 2. K25R, 3. L197A, 4. Y228E, 5. Y228F, 6. E271A, 7. D274A, 8. L283A, 9. L292A, 10. I313A, 11. I331A, 12. L342A). Control Immunopurifications of untransfected cells had no E2 protein, (data not shown). B. E2 (green) and AP50 (red), are co-localizationed (yellow) by confocal immunostaining. Nuclei are stained with TO-PRO3, (blue). C. Kinase assay of AP50 with E2, (lanes 1. Control (without E2), 2. HCV E2 wt, 3. K25R, 4. L197A, 5. Y228E, 6. Y228F, 7. E271A, 8. D274A, 9. L283A, 10. L292A, 11. I313A, 12. I331A, 13. L342A.).

FIG. 8 illustrates that mutations of the phosphorylation, cargo, and endocytic motifs of E2 disrupt its association with AP50 and HSC 70, and its auto-phosphorylation. A. The HCV E2 protein, (green) transfected into primary mouse hepatocytes is shown to co-localize (yellow) with AP50 by immunostaining with primary antibodies to HCV E2 and AP50. Nuclei are stained with TO-PRO3, (blue). B. In vitro Kinase assay of AP50 and E2. E2 autophosphorylation is shown (lanes 1. HCV E2 wt, 2. K25R, 3. L197A, 4. Y228E, 5. Y228F, 6. E271A, 7. D274A, 8. L283A, 9. L292A, 10. I313A, 11. I331A, 12. L342A.) C. The HCV E2 protein (green) transfected into primary mouse hepatocytes is shown to co-localize (yellow) with HSC 70 (red) by immunostaining with primary antibodies to HCV E2 and HSC 70. Nuclei are stained with TO-PRO3, (blue).

FIG. 9 illustrates that HCV E2 increases Clathrin HC expression and the endocytosis of Tf. A. Clathrin HC was immunopurified from above cells. (lanes 1. Control (without E2), 2. HCV E2 wt, 3. K25R, 4. L197A, 5. Y228E, 6. Y228F, 7. E271A, 8. D274A, 9. L283A, 10. L292A, 11. I313A, 12. I331A, 13. L342A). B. E2 (green) and Clathrin HC (red), are co-localized (yellow). Nuclei are stained with TO-PRO3, (blue). C. E2 increases the internalization of Tf as assayed by ¹²⁵I Tf. D. E2 decreases the internalization of EGF as was measured with ¹²⁵I EGF.

FIG. 10 illustrates that mutations in the kinase, cargo, or endocytic motifs of E2 disrupt its effect upon endocytosis. A. Reciprocal immunopurifications of E2 and immunoblots of Clathrin HC and E2 in primary mouse hepatocytes transfected as above (lanes 1. Control, 2. HCV E2 wt, 3. K25R, 4. L197A, 5. Y228E, 6. Y228F, 7. E271A, 8. D274A, 9. L283A, 10. L292A, 11. I313A, 12. I331A, 13. L342A). B. Primary mouse hepatocytes transfected with E2 were immunostained for E2 (green) and Clathrin HC (red), co-localization (yellow). Nuclei are stained with TO-PRO3, (blue). C. The internalization of ¹²⁵I Tf is not increased by the mutants as it is by E2 wt. Mutants L197A, Y228E, Y228F, E271A, L283A, I313A, I331A and L342A are notably deficient in their ability to internalize Tf. D. The surface binding of T-f assayed by ¹²⁵I Tf, shows no difference in Tf binding between control cells and cells with E2 wt. However, K25R, Y228E, Y228F, E271A, D274A, L283A, I331A, and L342A all have decreased surface Tf. E. The internalization of ¹²⁵I EGF by the E2 mutants is distinct from that of E2 wt. K2SR, I313A, I331A, and L342A mutants have a similar to E2, decrease in EGF internalization, but it is delayed. L197A, Y228E, Y228F, and D274A mutants all have an internalization of EGF similar to control, but also delayed. F. The surface binding of EGF assayed by ¹²⁵I EGF, shows no difference between control and E2 wt cells. K25R mutant shows a much lower surface binding that E2 wt or control. L197A, Y228, Y228F, E271A, D274A, L283A, L292A, I313A, I331A, and I342A all have greater surface binding that either E2 wt or control.

FIG. 11 illustrates that HCV E2 induces primary hepatocyte proliferation through the activation of the PI-3 kinase cascade, in the absence of external growth stimuli. A. E2 increased PIP2 as shown by an immunopurification/immunoblot (lanes 1. Control, 2. HCV E2 wt, 3. K25R, 4. L197A, 5. Y228, 6. Y228F, 7. E271A, 8. D274A, 9. L283A, 10. L292A, 11. I313A, 12. I331A, 13. L342A). B. PI-3 kinase expression and activity (phosphorylation) was increased by E2 shown in an immunopurification/immunoblot (cells and lanes, as above). C. HCV E2 increased Akt expression and activity shown in an immunopurification/immunoblot (cells and lanes, as above). D HCV E2 decreased BAD activity shown in an immunopurification/immunoblot (cells and lanes, as above E. DNA replication was measured by ³H thymidine incorporation (lanes 1 Control (without E2). 2. TGFa, 3. EGF, 4. E2 wt, 5. K25R, 6. L197A, 7. Y228E, 8. Y228F, 9. E271A, 10. D274A, 11. L283A, 12. L292A, 13. I313A, 14. I331A, 15. L342A).

FIG. 12 illustrates the structure of the dominant negative AP-50 peptide. The structure includes the dominant negative AP-50 with a T¹⁵⁶→A mutation (QGA156VQ), the 15-amino acid HIV-tat leading peptide and the fluorescein tag.

FIG. 13 illustrates that a dominant negative AP-50 peptide inhibits HCV infection in human hepatocyte cultures. A) The AP-50 peptide prevented phosphorylation of endogenous AP-50 by recombinant HCV E2, as determined by a cell-free kinase assay of purified AP-50 as described in Methods. The IC₅₀ was ˜150 μM. E2 was auto-phosphorylated. AP-50 was not phosphorylated in the absence of E2. B) Human hepatocytes were incubated with the cell permeable, AP-50 peptide for 72 hr, while infected with serum-derived HCV genotype 1 (56,000 virions). Control human hepatocytes were infected with HCV genotype 1 but incubated without the AP-50 peptide. The AP-50 peptide was intracellular as indicated by the green FITC fluorescence, and it was associated with HCV E2 (red) in the treated cells (merge). C) Confocal microscopy was performed for nuclei (TO-PRO-3; blue) and AP-50 phoshoT¹⁵⁶ (pAP-50; red) in control, uninfected (upper panels) and HCV-infected (lower panels) human liver. AP-50 phoshoT¹⁵⁶ is increased in the HCV-infected liver compared to control. D) Confocal microscopy was performed for nuclei (TO-PRO-3; blue), AP-50 phoshoT¹⁵⁶ (pAP-50; red) and AP-50 peptide (green) in control, uninfected (upper panels), HCV-infected (middle panels) and HCV-infected, treated with the AP-50 peptide (lower panels) human hepatocyte cultures. AP-50 phoshoT¹⁵⁶ is increased in the HCV-infected human hepatocyte cultures compared to control In the experiment described in (B), the AP-50 peptide blocked phosphorylation of endogenous AP-50 on Thr¹⁵⁶, as determined with epitope specific antibodies. E) The AP-50 peptide (90 pM) inhibited HCV replication of genotype 1, as detected by expression of HCV RNA in human hepatocyte cultures as described in (B). Results from triplicate samples of five independent experiments; P<1.01 for AP-50 peptide. F) The AP-50 peptide (90 μM) inhibited HCV replication of genotypes 1, 3 and 4, as detected by expression of HCV RNA at 72 hr in human hepatocyte cultures treated 4 hr after the HCV infection.

FIG. 14 illustrates that the AP-50 peptide is not toxic to human hepatocytes. Cells were treated with the AP-50 peptide for 72 hr. Cellular toxicity was determined by the release of lactic dehydrogenase (LDH) into the medium, and values are expressed relative to control samples. LDH values were increased in human hepatocytes infected with HCV genotypes 1, 3 or 4, but normalized in HCV-infected hepatocytes treated with the AP-50 peptide.

FIG. 15 illustrates that the phosphorylation mimic AP-50 peptide does not affect HCV infection. Human hepatocytes were incubated with the cell permeable, phosphorylation mimic AP-50 peptide for 72 h, while infected with serum-derived HCV genotype 1 as described in FIG. 4. The peptide is identical to that described in FIG. 4 but with E¹⁵⁶. Control human hepatocytes were infected with HCV genotype 1 but incubated without the phosphorylation mimic AP-50 peptide. The phosphorylation mimic AP-50 peptide (90 μM) did not affect HCV replication of genotype 1 as detected by expression of HCV RNA in human hepatocyte cultures. Results from triplicate samples of two independent experiments; P: NS.

### DETAILED DESCRIPTION OF THE INVENTION

The present invention provides an isolated compounds, peptides, antibodies, vaccine, preferably peptides, for inhibiting HCV infection comprising a peptide that inhibits one or more functional domains of HCV E2 protein from interacting with associated proteins selected from the group consisting of AP-50, HSC70, Cyclin A, and Cyclin G. In one preferred embodiment, the isolated peptide binds to an amino acid sequence of LIXXQXTG (SEQ ID NO:1). SGREYALKR (SEQ ID NO:32), or LVGLLTPGAKQNIQLI (SEQ ID NO:33) of the HCV E2 envelop protein. In yet another preferred embodiment, the isolated peptide is an AP-50 mutant. In yet another preferred embodiment, the isolated peptide comprises an AP-50 mutant comprising an amino acid sequence of QGAVQ (SEQ ID NO:2) having an Alanine substitution at position 156 (Ala156) of a native AR-50.

In yet another preferred embodiment, the isolated peptide comprises an AP-50 mutant that lacks a functional domain of a native AP-50. Preferably, the functional domain is a J domain of a native AP-50. In yet another preferred embodiment, the isolated peptide is mutant of HCV associated proteins including, but not limited to HCV E2, HSC70, Cyclin C, and Cyclin G, In yet another preferred embodiment, the isolated peptide comprises a HCV E2 mutant having less PI-3K activating capacity than native HCV E2 protein.

As used herein, the term “peptide” refers to a chain of at least three amino acids joined by peptide bonds. The term “peptide” and “protein” are use interchangeably. The chain may be linear, branched, circular, or combinations thereof. As used herein, the term “analogs” refers to two amino acids that have the same or similar function, but that have evolved separately in unrelated organisms. As used herein, the term “analog” further refers to a structural derivative of a parent compound that often differs from it by a single element. As used herein, the term “analog” also refers to any peptide modifications known to the art, including but are not limited to changing the side chain of one or more amino acids or replacing one or more amino acid with any non-amino acids.

In certain embodiments the peptides and analogs of the present invention are isolated or purified. Protein purification techniques are well known in the art. These techniques involve, at one level, the homogenization and crude fractionation of the cells, tissue or organ to peptide and non-peptide fractions. The peptides of the present invention may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. A particularly efficient method of purifying peptides is fast protein liquid chromatography (FPLC) or even HPLC.

An isolated peptide is intended to refer to a peptide/protein that is purified to any degree relative to its naturally-occurring state. Therefore, an isolated or purified peptide refers to a peptide free from at least some of the environment in which it may naturally occur. Generally, “purified” will refer to a peptide composition that has been subjected to fractionation to remove various other components, and which composition substantially retains its expressed biological activity. Where the term “substantially purified” is used, this designation will refer to a composition in which the peptide forms the major component of the composition, such as constituting about 50%, about 60%, about 70%, about 80%, about 90%, about 95%, or more of the peptides in the composition.

Various methods for quantifying the degree of purification of the peptide are known in the art. These include, for example, determining the specific activity of an active fraction, or assessing the amount of peptides within a fraction by SDS/PAGE analysis. Various techniques suitable for use in peptide/protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides and their analogs always be provided in their most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein. The invention contemplates compositions comprising the peptides and a pharmaceutically acceptable carrier.

In certain embodiments, the peptides and their analogs of the present invention may be attached to imaging agents including but are not limited to fluorescent, and/or radioisotopes including but are not limited to ¹²⁵I, for imaging, diagnosis and/or therapeutic purposes. Many appropriate imaging agents and radioisotopes are known in the art, as are methods for their attachment to the peptides.

The present invention also provides isolated nucleotides encoding the aforementioned proteins or peptides that are capable of interacting with HCV NS1/E2 envelop protein or AP-50 to disrupt and inhibit HCV infection. In one of the preferred embodiments, the present invention provides an isolated nucleotide encoding a peptide comprising an AP-50 mutant comprising an amino acid sequence as set forth in SEQ ID NO:2, In yet another preferred embodiment, the present invention provides an isolated nucleotide encoding a peptide comprising a dominant negative AP-50 mutant further comprising an Alanine substitution at position 156 (Ala156) of a native HCV E2 protein, In yet another preferred embodiment, the present invention provides an isolated nucleotide encoding a peptide comprising a HCV E2 mutant having less PI-3K activating capacity than native HCV E2 protein. In yet another preferred embodiment, the present invention provides an isolated nucleotide encoding a peptide comprising HSC70 protein, Cyclin A or Cyclin G protein, or mutants thereof.

As used herein, the “nucleic acids” or “nucleotides” may be derived from genomic DNA, complementary DNA (cDNA) or synthetic DNA. The term “nucleic acid” or “nucleotide” also refer to RNA or DNA that is linear or branched, single or double stranded, chemically modified, or a RNA/DNA hybrid thereof. It is contemplated that a nucleic acid within the scope of the present invention may comprise 3-100 or more nucleotide residues in length, preferably, 9-45 nucleotide residues in length, most preferably, 15-24 nucleotide residues in length. Where incorporation into an expression vector is desired, the nucleic acid may also comprise a natural intron or an intron derived from another gene. Less common bases, such as inosine, 5-methylcytosine, 6-methyladenine, hypoxanthine, and others can also be used.

An “isolated” nucleic acid molecule is one that is substantially separated from other nucleic acid molecules which are present in the natural source of the nucleic acid (i.e., sequences encoding other polypeptides). Preferably, an “isolated” nucleic acid is free of some of the sequences which naturally flank the nucleic acid (i.e., sequences located at the 5′ and 3′ ends of the nucleic acid) in its naturally occurring replicon, For example, a cloned nucleic acid is considered isolated. A nucleic acid is also considered isolated if it has been altered by human intervention, or placed in a locus or location that is not its natural site, or if it is introduced into a cell by agroinfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

As used herein, “homologs” are defined herein as two nucleic acids or peptides that have similar, or substantially identical, nucleic acids or amino acid sequences, respectively. The term “homolog” further encompasses nucleic acid molecules that differ from one of the nucleotide sequences due to degeneracy of the genetic code and thus encodes the same amino acid sequences. In one of the preferred embodiments, homologs include allelic variants, orthologs, paralogs, agonists, and antagonists of nucleic acids encoding the peptide, or analogs thereof, of the present invention.

As used herein, the term “orthologs” refers to two nucleic acids from different species, but that have evolved from a common ancestral gene by speciation. Normally, orthologs encode peptides having the same or similar functions. In particular, orthologs of the invention will generally exhibit at least 80-85%, more preferably 85-90% or 90-95%, and most preferably 95%, 96%, 97%, 98%, or even 99% identity, or 100% sequence identity, with all or part of the amino acid sequence of the peptides, or analogs thereof, of the present invention, preferably, SEQ ID NO:2, or mutants thereof and will exhibit a function similar to these peptides. Preferably, the orthologs of the present invention associate with HCV E2 protein and function as HCV E2 inhibitors and/or modulators. As also used herein, the term “paralogs” refers to two nucleic acids that are related by duplication within a genome. Paralogs usually have different functions, but these functions may be related (Tatusov et al., 1997, Science 278(5338):631-637).

To determine the percent sequence identity of two amino acid sequences (e.g., SEQ ID NO:2, and a mutant form thereof, the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence (e.g., SEQ ID NO:2) is occupied by the same amino acid residue as the corresponding position in the other sequence (e.g., a mutant form of the sequence selected from the peptide sequences of SEQ ID NO:2), then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). Preferably, the isolated amino acid homologs included in the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence shown in SEQ ID NO:2, or mutant thereof, In one preferred embodiment, the isolated nucleic acid homologs of the present invention encode amino acid sequence of SEQ ID NO:2, or portion thereof, that is at least 90%, more preferably at least 95% identical to an amino acid sequence of SEQ ID NO:2, and associate with HCV E2 protein, regulating AP-50 protein phosphorylation. In yet another preferred embodiment, the isolated nucleic acid homologs of the present invention encode amino acid sequence, or portion thereof, that is at least 90%, more preferably at least 95% identical to an amino acid sequence of a HCV E2 protein, AP-50 protein, HSC70 protein, Cyclin A, Cyclin G, or mutants thereof.

The determination of the percent sequence identity between two nucleic acid or peptide sequences is well known in the art. For instance, the Vector NTI 6.0 (PC) software package (InforMax, 7600 Wisconsin Ave., Bethesda, Md. 20814) to determine the percent sequence identity between two nucleic acid or peptide sequences can be used, In this method, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nueleotide.

In another aspect, the present invention provides an isolated nucleic acid comprising a nucleotide sequence that hybridizes to the nucleotides encoding the amino acid sequences shown in SEQ ID NO:2 under stringent conditions. In yet another aspect, the present invention provides an isolated nucleic acid comprising a nucleotide sequence that hybridizes to the nucleotides encoding the amino acid sequences of a HCV E2 protein, AP-50 protein, HSC70 protein, Cyclin A, Cyclin G, or mutants thereof, of the invention, under stringent conditions.

As used herein with regard to hybridization for DNA to a DNA blot, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10× Denhart's solution, 6×SSC, 0.5% SDS and 100 μg/ml denatured salmon sperm DNA. Blots are washed sequentially at 65° C. for 30 minutes each time in 3×SSC/0.1% SS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al., eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, N.Y., 1993.

Using the above-described methods, and others known to those of skill in the art, one of ordinary skill in the art can isolate homologs of the peptides of the present invention comprising the amino acid sequence shown in SEQ ID NO:2, or mutant thereof. In yet another preferred embodiment, one of ordinary skill in the art can also isolate homologs of the peptides of the present invention comprising an amino acid sequence of a HCV E2 protein, AP-50 protein, Cyclin A protein, Cyclin G protein, or mutants thereof. One subset of these homologs are allelic variants. As used herein, the term “allelic variant” refers to a nucleotide sequence containing polymorphisms that lead to changes in the amino acid sequences of the peptides of the present invention without altering the functional activities. Such allelic variations can typically result in 1-5% variance in nucleic acids encoding the peptides of the present invention (e.g., SEQ ID NO:2, or mutant thereof).

In addition, the skilled artisan will further appreciate that changes can be introduced by mutation into a nucleotide sequence that encodes the amino acid sequence of the peptides, or analogs thereof, of the present invention (e.g., SEQ ID NO:2). For example, nucleotide substitutions leading to amino acid substitutions at “non-essential” amino acid residues can be made in a sequence encoding the amino acid sequence of the peptides, or analogs thereof, of the present invention. A “non-essential” amino acid residue is a residue that can be altered without altering the activity of said peptide, whereas an “essential” amino acid residue is required for desired activity of such peptide, such as enhance or facilitate transdermal delivery of any drugs.

In one embodiment, the isolated nucleic acid molecule comprises a nucleotide sequence encoding a peptide, wherein the peptide comprises an amino acid sequence at least about 50% identical to an amino acid sequence of SEQ ID NO:2, or mutants thereof. Preferably, the peptide encoded by the nucleic acid molecule is at least about 50-60% identical to an amino acid sequence of SEQ ID NO:2, or mutant thereof, more preferably at least about 60-70% identical, even more preferably at least about 70-75%, 75-80%, 80-85%, 85-90%, or 90-95% identical, and most preferably at least about 96%, 97%, 98%, or 99% identical to an amino acid sequence of SEQ ID NO:2, or mutants thereof.

In yet another preferred embodiment, the isolated nucleic acid molecule comprises a nucleotide sequence encoding a peptide, wherein the peptide comprises an amino acid sequence at least about 50% identical to an amino acid sequence of a HCV E2 peptide, AP-50 peptide, Cyclin A peptide, Cyclin G peptide, or mutants thereof. Preferably, the peptide encoded by the nucleic acid molecule is at least about 50-60% identical to an amino acid sequence of SEQ ID NO:2, or mutant thereof more preferably at least about 60-70% identical, even more preferably at least about 70-75%, 75-80%, 80-85%. 85-90%, or 90-95% identical, and most preferably at least about 96%, 97%, 98%, or 99% identical to an amino acid sequence of a HCV E2 peptide, AP-50 peptide, Cyclin A peptide, Cyclin G peptide, or mutants thereof.

An isolated nucleic acid molecule encoding the peptides of the present invention can be created by introducing one or more nucleotide substitutions, additions, or deletions into a nucleotide encoding the peptide sequence, such that one or more amino acid substitutions, additions, or deletions are introduced into the encoded peptide and/or the side chain of the amino acids constituting the encoded peptides. Mutations can be introduced into the nucleic acid sequence encoding the peptide sequence of the present invention by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Following mutagenesis of the nucleic acid sequence encoding the peptides of the present invention, the encoded peptide can be expressed recombinantly and the activity of the peptide can be determined by analyzing interaction with HCV E2 protein.

The nucleotides of the present invention may be produced by any means, including genomic preparations, cDNA preparations, in vitro synthesis, RT-PCR, and in vitro or in vivo transcription. It is contemplated that peptides of the present invention, their variations and mutations, or fusion peptides/proteins may be encoded by any nucleic acid sequence that encodes the appropriate amino acid sequence. The design and production of nucleic acids encoding a desired amino acid sequence is well known to those of skill in the art based on standardized codons. In preferred embodiments, the codons selected for encoding each amino acid may be modified to optimize expression of the nucleic acid in the host cell of interest. Codon preferences for various species of host cell are well known in the art.

Any peptides and their analogs comprising the isolated peptides of the present invention can be made by any techniques known to those of skill in the art, including but are not limited to the recombinant expression through standard molecular biological techniques, the conventional peptide/protein purification and isolation methods, and/or the synthetic chemical synthesis methods. The nucleotide and peptide sequences corresponding to various genes may be found at computerized databases known to those of ordinary skill in the art, for instance, the National Center for Biotechnology Information's Genbank and GenPept databases National Center for Biotechnology Information). Alternatively, various commercial preparations of proteins and peptides are known to those of skill in the art.

Because the length of the isolated peptides of the present invention is relatively short, peptides and analogs comprising the amino acid sequences of these isolated peptide inserts can be chemically synthesized in solution or on a solid support in accordance with conventional techniques. Various automatic synthesizers are commercially available and can be used in accordance with known protocols. Short peptide sequences, usually from about 5 up to about 35 to 50 amino acids, can be readily synthesized by such methods. Alternatively, recombinant DNA technology may be employed wherein a nucleotide sequence which encodes a peptide and its analog of the present invention is inserted into an expression vector, transformed or transfected into an appropriate host cell, and cultivated under conditions suitable for expression.

Peptide mimetics may also be used for preparation of the peptides and their analogs of the present invention. Mimetics are peptide-containing molecules that mimic elements of protein secondary structure. A peptide mimetic is expected to permit molecular interactions similar to the natural molecule, and may be used to engineer second generation molecules having many of the natural properties of the peptides, but with altered and even improved characteristics.

The present invention also provides chimeric or fusion peptides that comprise the amino acid sequences of the isolated peptides of the present invention, as disclosed herein. As used herein, a “chimeric or fusion peptide” comprises the amino acid sequence corresponding to the amino acid sequence of the peptides, or analogs thereof, of the present invention, operatively linked, preferably at the N- or C-terminus, to all or a portion of a second peptide or protein. As used herein, “the second peptide or protein” refer to a peptide or protein having an amino acid sequence which is not substantially identical to the amino acid sequences of the peptides, analogs, or mutants thereof, of the present invention, e.g., a peptide or protein that is different from HCV E2 protein, AP-50 protein, Cyclin A protein, Cyclin G protein, or analogs thereof, and is derived from the same or a different organism. With respect to the fusion peptide, the term “operatively linked” is intended to indicate that the amino acid of the peptides, or analogs thereof, of the present invention, and the second peptide or protein are fused to each other so that both sequences fulfill the proposed function attributed to the sequence used.

For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the present invention comprise the peptide and/or analog comprising amino acid sequences of the displayed peptide identified from the in vivo phage display, that is linked to a therapeutic protein or peptide. Examples of proteins or peptides that may be incorporated into a fusion protein include cyrtostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually any protein or peptide could be incorporated into a fusion protein comprising the peptides and analogs of the present invention. Furthermore, in certain preferred embodiments, the fusion proteins of the present invention exhibit enhanced transdermal penetration capability as compared to non-fusion proteins or peptides that have not fused with the peptides and analogs, as disclosed herein.

Methods of generating fusion peptides/proteins are well known to those of skill in the art. Such peptides/proteins can be produced, for example, by chemical attachment using bifunctional cross-linking reagents, by de novo synthesis of the complete fusion peptide/protein, or by standard recombinant DNA techniques that involve attachment of a DNA sequence encoding the peptides of present invention, as disclosed herein, to a DNA sequence encoding the second peptide or protein, followed by expression of the intact fusion peptide/protein using. For example, DNA fragments coding for the peptide sequences of the peptides, or analogs thereof, of the present invention, are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (See, for example, Current Protocols in Molecular Biology, Eds. Ausubel et al., 1992, John Wiley & Sons). Moreover, many expression vectors are commercially available that already encode a fusion moiety (e.g., a GST polypeptide). The nucleic acids encoding peptides, analogs, or mutants thereof, of the present invention can be cloned into such an expression vector such that the fusion moiety is linked in-frame to these nucleic acids encoding peptides, or analogs or mutants thereof, of the present invention.

The present invention further provides a pharmaceutical composition for preventing and/or treating HCV infection comprising the isolated peptides, mutants, or analogs thereof of the present invention and any pharmaceutically acceptable excipients. Pharmaceutically acceptable excipients are well known in the art, and have been amply described in variety of publications, including, for example, “Remington: The Science and Practice of Pharmacy”, 19^{th} Ed. (I 995).

The present invention further comprises methods for preventing or treating HCV infection comprising administering to a subject at need an effective amount of pharmaceutical composition comprising the isolated peptides, mutants, or analogs thereof, of the present invention. In preferred embodiments, the isolated peptides, mutants, or analogs thereof, can be used as a therapeutic agent for treating HCV infection. As used herein, the term “therapeutic agent,”, or “drug” is used interchangeably to refer to a chemical material or compound that inhibit HCV infection.

In yet another preferred embodiment, the isolated peptides, mutants, analogs thereof, of the present invention can also be incorporated into vectors/virus and used for gene therapy. The term “gene therapy” refers to a technique for correcting defective genes responsible for disease development. Such techniques may include inserting a normal gene into a nonspecific location within the genome to replace a nonfunctional gene; swapping an abnormal gene for a normal gene through homologous recombinations, reparing an abnormal gene to resume its normal function through selective reverse mutation; and altering or regulating gene expression and/or functions of a particular gene. In most gene therapy, a normal gene is inserted into the genome to replace an abnormal or disease-causing gene.

As used herein, a term “vector/virus” refers to a carrier molecule that carries and delivers the “normal” therapeutic gene to the patient's target cells. Because viruses have evolved a way of encapsulating and delivering their genes to human cells in a pathogenic manner, most common vectors for gene therapy are viruses that have been genetically altered to carry the normal human DNA. As used herein, the viruses/vectors for gene therapy include retroviruses, adenoviruses, adeno-associated viruses, and herpes simplex viruses. The term “retrovirus” refers to a class of viruses that can create double-stranded DNA copies of their RNA genomes, which can be further integrated into the chromosomes of host cells, for example, Human immunodeficiency virus (HIV) is a retrovirus. The term “adenovirus” refers to a class of viruses with double-stranded DNA genomes that cause respiratory, intestinal, and eye infections in human, for instance, the virus that cause the common cold is an adenovirus. The term “adeno-associated virus” refers to a class of small, single-stranded DNA viruses that can insert their genetic material at a specific site on chromosome 19. The term “herpes simplex viruses” refers to a class of double-stranded DNA viruses that infect a particular cell type, neurons. Herpes simplex virus type 1 is a common human pathogen that causes cold sores.

The present invention further provides antibodies and vaccines generated from, and/or comprising the isolated peptides of the present invention for HCV prevention and/or treatment. The term “antibody” includes complete antibodies, as well as fragments thereof (e.g., F(ab′)2, Fab, etc.) and modified antibodies produced therefrom (e.g., antibodies modified through chemical, biochemical, or recombinant DNA methodologies), with the proviso that the antibody fragments and modified antibodies retain antigen binding characteristics sufficiently similar to the starting antibody so as to provide for specific detection of antigen.

Antibodies may be prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

As used herein, the term “vaccine” refers to a product that produces immunity therefore protecting the body from the disease. Vaccines that comprise a suspension of attenuated or killed microorganism (e.g. bacterial, viruses, or) are administered for the prevention, amelioration or treatment of infectious diseases. In preferred embodiments, the present invention provides HCV vaccines generated from, and/or comprising the isolated peptide, mutants, or analogs thereof of the present invention.

As used herein, the terms “treatment,” “treating,” and the like, refer to obtaining a desired pharmacologic and/or physiologic effect. The effect may be prophylactic in terms of completely or partially preventing a symptom thereof and/or may be therapeutic in terms of a partial or complete cure for an adverse affect attributable to the condition. “Treatment,” as used herein, covers any treatment of an injury in a mammal, particularly in a human, and includes: (a) preventing HCV infection, arresting any complications, and minimizing its effects; (b) relieving the symptoms; (c) preventing the disease from occurring in a subject which may be predisposed to the disease but has not yet been diagnosed as having it; (d) inhibiting the disease, i.e., arresting its development; and (e) relieving the disease, i.e., causing regression of the disease.

As used herein, the term “individual,” “host,” “subject.” and “patient” are used interchangeably herein, and refer to a mammal, including, but not limited to, murines, simians, humans, mammalian farm animals, mammalian sport animals, and mammalian pets.

As used herein, the term “effective amount” or “therapeutically effective amount” means a dosage sufficient to provide treatment of the disease state being treated or to otherwise provide a desired pharmacologic and/or physiologic effect.

The present invention also provides a primary hepatocyte cell culture comprising hepatocytes derived from a healthy subject and a bodily fluid derived from a HCV infected subject. In one preferred embodiment, the bodily fluid is serum or plasma, preferably serum, in yet another preferred embodiment, the primary hepatocyte cell culture of comprisese HCV genotypes 1, 2, 3, 4, or combinations thereof. In yet another preferred embodiment, the subject is a human.

The human primary hepatocyte cell culture of the present invention provides a great potential in studying the mechanism of infection by HCV, and further provides insights into ways to inhibit the infection process. In one preferred embodiment, the present invention provides that HCV E2 and core proteins persists throughout weeks in the cell culture system of the present invention. In yet another preferred embodiment, the present invention provides that HCV produced in the cell culture system of the present invention is further infectious to Naïve primary human hepatocytes. The present invention further provides that the human primary hepatocyte cell culture system provides a greater predictive value for evaluating drug responses with respect to their efficacy and toxicology in treating HCV infection, and can be used in special patient populations.

The present invention further provides a method for screening a compound for inhibiting HCV infection. Such method comprises a) obtaining the primary hepatocyte cell culture of claim 22, b) infecting said primary hepatocyte cell culture with HCV in the absence or presence of said compound, and c) determining differences of HCV infection in the cultures in the absence or presence of said compound.

In yet another preferred embodiment, the level of HCV infection can be determined by determining and calculating HCV particles in electron microscopy, In another preferred embodiment, the level of HCV infection can be determined by determining and calculating HCV virons by quantitative PCR or radio labeling techniques, or by taking viral titers. In yet another preferred embodiment, the level of HCV infection can be determined by determining and calculating HCV related proteins and cellular structures and/or pathways in the cell culture system of the present invention. Preferably, the HCV related proteins being determined in the cell culture system of the present invention include but are not limited to HCV E2 proteins and HCV core proteins, In yet another preferred embodiment, all HCV genotypes (e.g., genotypes 1-4) can be determined in the cell culture system of the present invention. In yet another preferred embodiment, the present invention provides that the HCV RNA replication of genotypes 1, 2, and 3 in infected primary human hepatocytes in the cell culture of the present invention is comparable at 48 hrs. to HCV infected liver.

These and many other variations and embodiments of the invention will be apparent to one of skill in the art upon a review of the appended description and examples.

### EXAMPLES

### Example 1

### Generating Human Primary Hepatocyte/HCV Infection Culture System with Human HCV-Position Sera

### Human HCV-Positive Sera

Sera from 25 HCV-infected patients and 3 control subjects were obtained at the VA San Diego Healthcare System Clinical Laboratory. The subject population included individuals with chronic HCV infection, viral load>200,000 IU/ml and genotypes 1 (N: 15), 2 (N: 3), 3 (N: 4), or 4 (N: 3), but negative for Hepatitis A and B, CMV and HIV. Control sera were obtained from subjects negative for Hepatitis A, B and C, CMV and HIV. In different experiments, the inoculums fluctuated between 3,728 and 68,000 HCV viral particles.

### Human Primary Hepatocyte Cultures

Hepatocytes (from Tissue Transformation Technologies [Edison, N.J.]) were obtained from anonymous organ donors without liver disease that were not suitable for liver transplantation for technical but not medical reasons. These donors were negative for Hepatitis A, B and C, CMV, HIV, HTLV ½, and RPR-STS. Hepatocytes cultures with >5% apoptosis by annexin-V assays and/or increases>3-fold in ALT were discarded, Hepatocytes were isolated from an encapsulated liver sample by a modified two-step perfusion technique introduced by Seglen (Methods Cell Biol 13, 29 (1976)). Briefly, the dissected lobe was placed into a custom-made perfusion apparatus and two to five hepatic vessels were cannulated with tubing attached to a multi-channel manifold. A liver fragment (150 to 500 g) was perfused initially (recirculation technique) with calcium-free HBSS supplemented with 0.5 mM EGTA for 20 to 30 min and then with 0.05% collagenase [Sigma] dissolved in L-15 medium (with calcium) at 37° C. until the tissue was fully digested. The digested liver was removed, immediately cooled with ice-cold L-15 medium and the cell suspension was strained through serial progressively smaller stainless steel sieves, with a final filtration through 100-micron and 60-micron nylon mesh. The filtered cell suspension was aliquoted into 250-ml tubes and centrifuged three times at 40 g for 3 min at 4° C. After the last centrifugation, the cells were re-suspended, in HypoThermosol-FRS [BioLife Solutions, Inc] combined in one tube and placed on ice.

Cells were centrifuged at 700 rpm for 5 min at 4° C., the supernatant was removed and the cells were washed with Hanks Wash Solution (53.6 mM KCl 0.4 g/l; 4.4 mM KH2PO 0.06 g/l; 1.37M NaCl 8 g/l; 3.4 mM, Na2HPO4 0.048 g/l; 20 mL CaCl2 (2M)) three times. Cells were re-suspended in Hepatocyte Plating Media (500 mL DMEM high glucose; 20% FBS) and plated at a concentration of at 0.625×106 cells/mL. Diluted collagen (type 1, rat tail-BD Cat. #354236) (50 ug/ml in 0.02N acetic acid) was used for coating coverslips and plates in 10 ml at room temperature for one hour. The collagen solution was then removed and rinsed once with PBS. After the cells attached (<18 hrs), the HPM was replaced by Hepatocyte Media (500 mL DMEM high glucose; 30 mg L-methionine; 104 mg L-leucine; 33.72 mg L-ornithine; 200 μL of 5 mM stock dexamethasone; 3 mg Insulin).

### Hepatocyte Culturing Conditions for Serum-Derived HCV Infection

The hepatocytes were cultured for serum-derived HCV infection under the following conditions: 1) the matrix was rat-tail collagen (BD); 2) the collagen matrix was prepared within 24 hr of hepatocyte plating, at a concentration of 50 μg/ml or greater; 3) the culture plates were coated with polylysine; 4) the rinsing of the matrix was minimal; 5) the suspended hepatocytes were allowed to attach in 20% fetal calf serum for not more than 18 hr; 6) the hepatocyte-specific media was given for at least 24 hr prior to the HCV infection; 7) the hepatocytes were >85% confluent until the time of infection; 8) hepatocyte cultures with >5% apoptosis by annexin-V assays and/or increases>3-fold in ALT were discarded; and 9) hepatocyte media was added every 48 hr.

### Example 2

### HCV Amplification in Human Primary Hepatocyte/HCV Infection Culture System

### Confocal Microscopy

Fluorescent labels were observed using a triple-channel fluorescence microscope or a confocal microscope. Fluorochromes utilized included TOPRO-3 (blue), Alexa 488 (green) and Alexa 594 (red) (Molecular Probes). The percentage of HCV infected hepatocytes was determined by confocal microscopy using HCV E2 and core specific antibodies (Buck, et al., Mol. Cell. 8, 807 (2001); Rudel et al., Science 276, 1571 (1997). AP-50 and AP-50-phosphoT¹⁵⁷ were detected with specific antibodies (Zhang et al., Traffic 6, 1103 (2005); Smythe, Nature 431, 641 (2004)). At least 100 cells were analyzed per experimental point (Buck et al. EMBO J 20, 6712 (2001)). The nuclear morphology were analyzed by staining cells with TOPRO-3 (R&D Systems). Two observers analyzed each immunofluorescent study.

### Transmission Electron Microscopy

Human hepatocytes cultures grown on chamber slides were fixed in modified Karnovsky's fixative (2% paraformaldehyde, 1% glutaraldehyde, 5 mM CaCl2 in 0.1M Na Cacodylate buffer, pH 7.4) overnight at 4 C followed by 1% OsO4 in 0.1M Na Cacodylate buffer, pH 7.4, en bloc staining with 4% uranyl acetate in 50% ethanol, and subsequently dehydrated using a graded series of ethanol solutions followed by a rinse with a 1:1 (v:v) mixture of 100% ethanol: propylene oxide and infiltration with epoxy resin (Scipoxy 812, Energy Beam Sciences, Agawam, Mass.). After polymerization at 65 C overnight, the slides were removed from the oven and the plastic slides were immediately peeled off the chambers leaving the cultures as a monolayer on the bottom of the chambers. Areas for thin sections were them cut from each chamber and mounted on blank chucks for sectioning. Thin sections were cut from the monolayer and stained with uranyl acetate (4% uranyl acetate in 50% ethanol) followed by bismuth subnitrate. Sections were examined at an accelerating voltage of 60 kV using a Zeiss EM108-C electron microscope at the Core Microscopy Facility VASDHS. For assessing the number of HCV virions per hepatocyte, 100 randomly selected cells per field, from 3 fields were analyzed per each point.

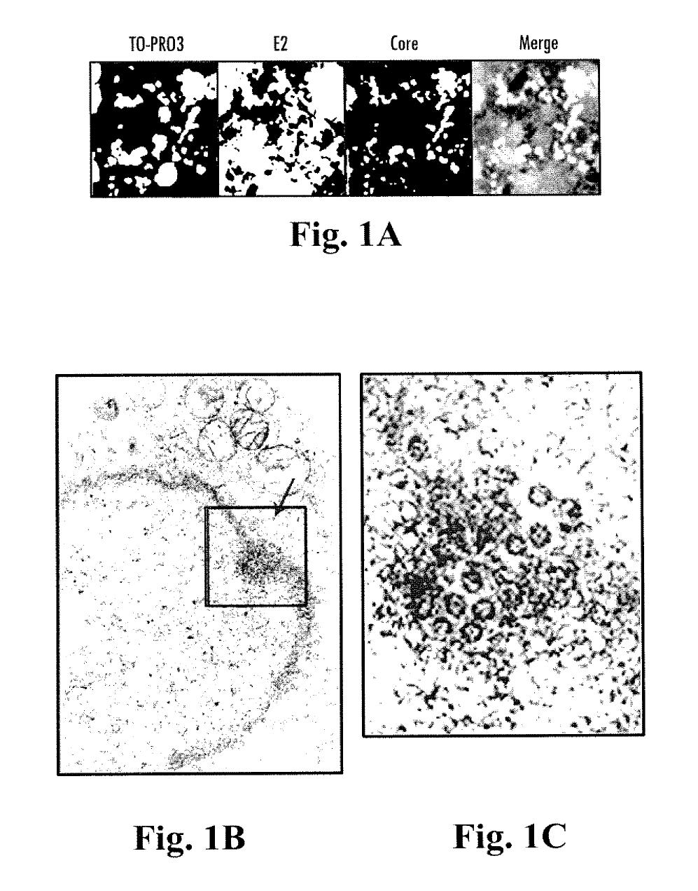

Serum-derived HCV infection of the hepatocyte occurred rapidly as reflected by the expression of HCV glycoprotein E2 and core proteins in the cell layers. FIG. 1A shows the expression of HCV glycoprotein E2 and core proteins on laser scanning confocal microscopy. Uninfected control hepatocytes were shown as background fluorescence in FIG. 1A. HCV glycoprotein E2 and core proteins co-localized in the perinuclear region of the hepatocytes infected with serum-derived HCV. By transmission electron microscopy, enveloped, virus-like structures that were localized to the perinuclear region of the hepatocytes were detected as shown in FIGS. 1B and 1C. These particles closely resembled in appearance and localization in the previously reported HCV virions in the liver of HCV-infected patients and chimpanzees (Vos et al., J Hepatol 37, 370 (2002); Shimizu, et al., Hepatology 23, 205 (1996)) and in the media of Huh-7 cells expressing a genomic HCV replicon (Heller et al, Proc, Natl. Acad. Sci. USA 102, 2579 (2005)). Table 1 shows greater than 95% of the cells contained HCV viral particles after a 24-hr exposure, indicating a robust HCV infection.

TABLE 1
HCV amplification in human hepatocyte culture

| Time [hr] | HCV virions/0⁶ hepatocytes | HCV virions/hepatocyte |
|---|---|---|
| 0 | 11.2 × 10³ | 1 × 10⁻² |
| (inoculum) | | |
| 4 | 1.2 × 10⁷ | 12 |
| 24 | 7.0 × 10⁷ | 70 |

Day-3, primary human hepatocytes were infected with HCV genotype 1 (11,200 HCV virions) for up to 24 hr.

Under the conditions described above, human hepatocyte cultures remained infected for at least 3 weeks. Because about 60% of hepatocytes had an average of >20 virions per cell after a 4-hr infection, and >95% of hepatocytes had an average of >75 virions per cell at 24-hr infection, the total hepatocyte viral load was about 12 million and 70 million, reflecting approximately a 1,000- and a 5.000-fold amplification at 4 hr and 24 hr, respectively, from the initial inoculum (Table 1). Table 1 also shows that an exponential HCV amplification occurred within the first 24 hr after infection. Further, the estimated HCV amplification in cultured human hepatocytes during the first 24 hr (˜70 HCV virions/hepatocyte/day) was at least as robust as the calculated HCV amplification in patients (˜10¹² HCV virions/day) (A. Neumann et al., Science 282, 103 (1998), when corrected by hepatocyte number in the human liver (˜10¹¹ hepatocytes) (H. Imamura et al., Hepatology 14, 448 (1991)) and in the culture system (Table 1).

### Example 3

### Detection of HCV Virons in the Human Primary Hepatocyte/HCV Infection Culture System

### Affinity Column Chromatography

Catch and Release affinity columns and protocol (Upstate) were used with HCV E-2 antibodies (Biodesign) with non-denaturing buffers as specified by the manufacturer. This method was more efficient and specific in purifying HCV virions than the standard immunoprecipitation techniques Negative and positive control samples were run in parallel.

### HCV RNA Determination

Total RNA was isolated from HCV infected primary human hepatocytes using the Ultraspec TM-II RNA kit from Biotecx Inc. (Texas) following the manufacturers protocol. The cDNA synthesis was performed using Stratascript RT MMuLV RNAse H free and Stratascript RT buffer (Stratagene), per manufacturers protocol, using:

HCV-Primer-A:

(SEQ ID NO: 3) for genotypes 1, 3 or 4;

AATTTAATACGACTCACTATAGGGACCTCGCAAGCACCCTATCAGGC
AGT
and
HCVg2aPrimer-A:

(SEQ ID No: 4) for genotype 2a.

AATTTAATACGACTCACTATAGGGACCTCGCAAGCGCCCTATCAGGC
AGT

PCR was performed on the cDNA using the cDNA reaction mixture with Qiagen's HotStar High Fidelity polymerase. Amplicons were run on 2% TBE agarose gels and imaged on a KODAK Imaging station.

The primers used were:

HCV-primer-A:

(SEQ ID NO: 5)

AATTTAATACGACTCACTATAGGGACCTCGCAAGCACCCTATCAGGCAG
T:.
HCV-Primer-B:

(SEQ ID NO: 6)

GCAGAAACCGTCTAGCCATGGCGT
HCVg2a-primer-A:

(SEQ ID NO: 7)

AATTTAATACGACTCACTATAGGGACCTCGCAAGCGCCCTATCAGGCAGT
HCVg2a-Primer-B:

(SEQ ID NO: 8)

GCAGAAAGCGCCTAGCCATGGCGT
HCVg3a-Primer-B:

(SEQ ID NO: 9)

GCGGAAAGCGCCTAGCCATGGCGT

Infection of Naïve Human Hepatocyte Cultures with Human Hepatocyte Culture-Derived HCV

Naïve day-3 primary human hepatocytes were cultured with 20 μl of cell layer lysates (estimated to be comparable to the original inoculum) from HCV-infected human hepatocytes cultures. HCV RNA was determined on infection day-3 as described above. In other experiments naïve human hepatocyte were cultured in a methionine-free medium for 72 hr. After this period, hepatocytes were infected as above, but in the presence of 100 μCi[³⁵S]-methionine (>1,000Ci/mMol) (MP Biomedicals). HCV E2 was immunopurified from cell layers, immunoblot and the E2 bands were excised and counted using a Beckman LS 6500 liquid scintillation counter.

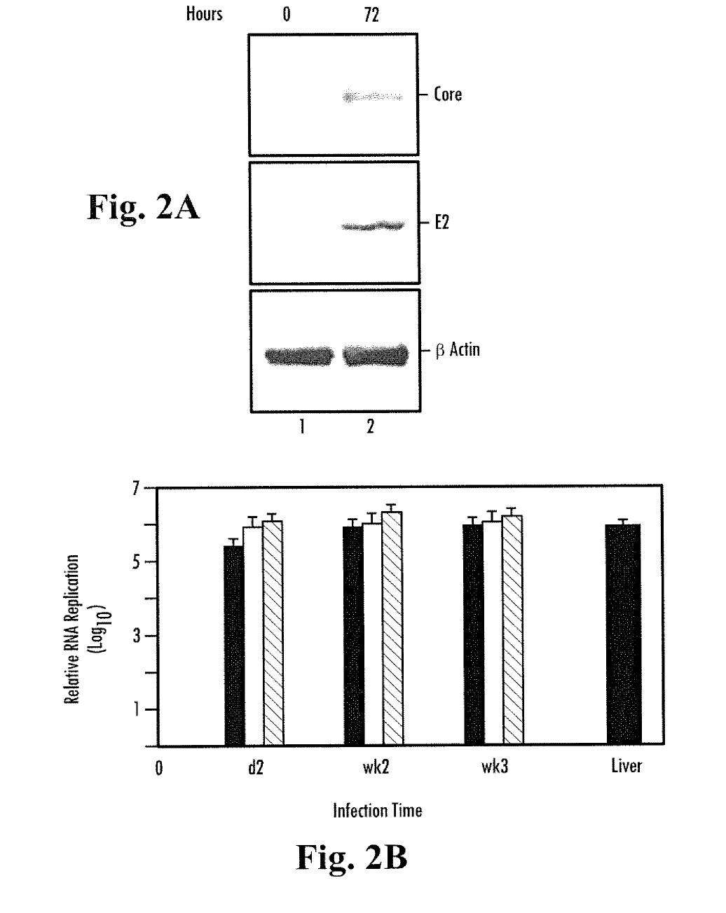

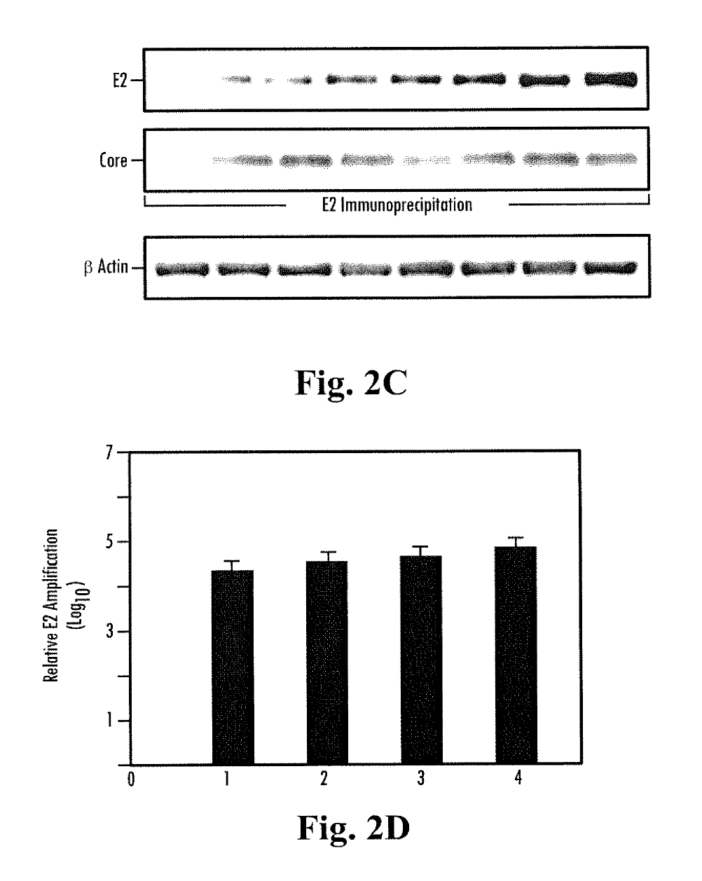

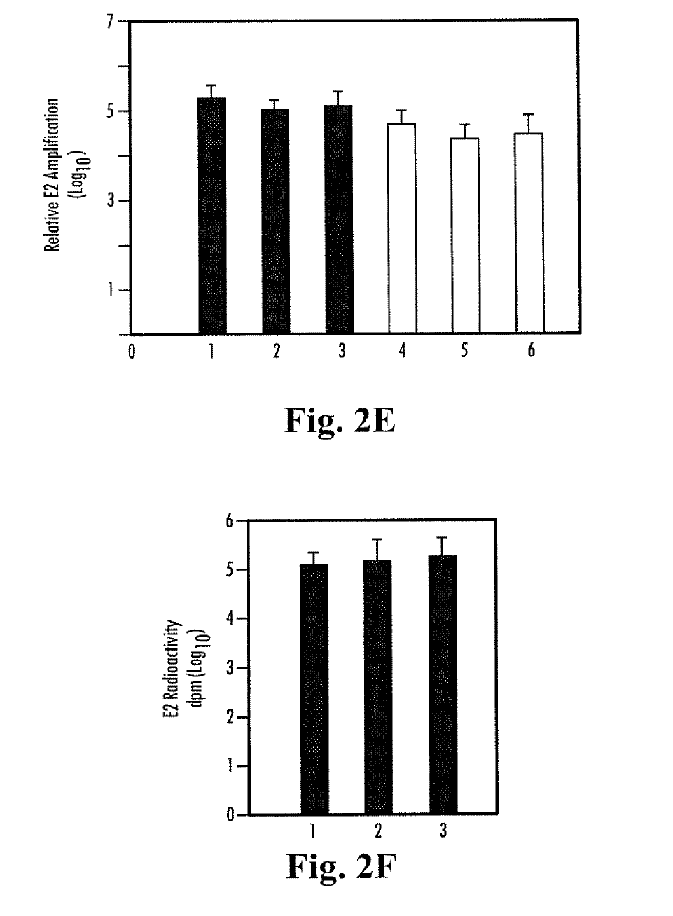

The amplification of HCV genotype 1 infection was analyzed by immunopurifying HCV virions from the medium through HCV E2 affinity chromatography. The HCV amplification was robust judging by the increased HCV E2 and core in the medium from time zero (inoculum) to 72 hr (FIG. 2A). Control samples from uninfected hepatocytes lacked detectable HCV E2 or core proteins. The infection-replication cascade was assessed by determining HCV genotype 1 viral particles in the hepatocyte culture from time zero to week-3. The HCV RNA increased exponentially up to day-2 infection, and it remained at that level for up to week-3 (FIG. 2B). The HCV RNA, corrected by total RNA, was comparable in human hepatocytes after day-2 and in the liver of HCV-infected patients (FIG. 2B). These data further support the validity of the human hepatocyte system to study HCV infection.

Moreover, a similar HCV RNA (FIG. 2B) and HCV E2 (FIGS. 2C and 2D) expression was detected in human hepatocytes infected with serum-derived HCV genotypes 1, 2, 3 and 4 obtained from patients chronically infected with HCV, indicating a consistent HCV infection of the human hepatocyte culture system for up to 3 weeks. Further, the medium HCV virions were infectious to naïve human hepatocyte cultures judging by the viral amplification as determined by immunopurification (FIG. 2E) or radioactive labeling (FIG. 2F) of newly synthesized HCV virions. The infectivity of serum-derived and human hepatocyte culture-produced HCV virions was comparable (FIG. 2E).

### Example 4

### HCV glycoprotein E2 and Its Association with AP-50 in the Human Primary Hepatocyte/HCV Infection Culture System

### Immunoprecipitation and Immunoblotting

HCV E2, HCV core, AP-50 and β-actin were detected by immunoblotting the immunoprecipitates from hepatocyte lysates as described (M. Buck et al., EMBO J. 13, 851 (1994)) following the chemiluminescence protocol (DuPont) and using purified IgG antibodies as described (C. Trautwein et al., Nature 364, 544 (1993)).

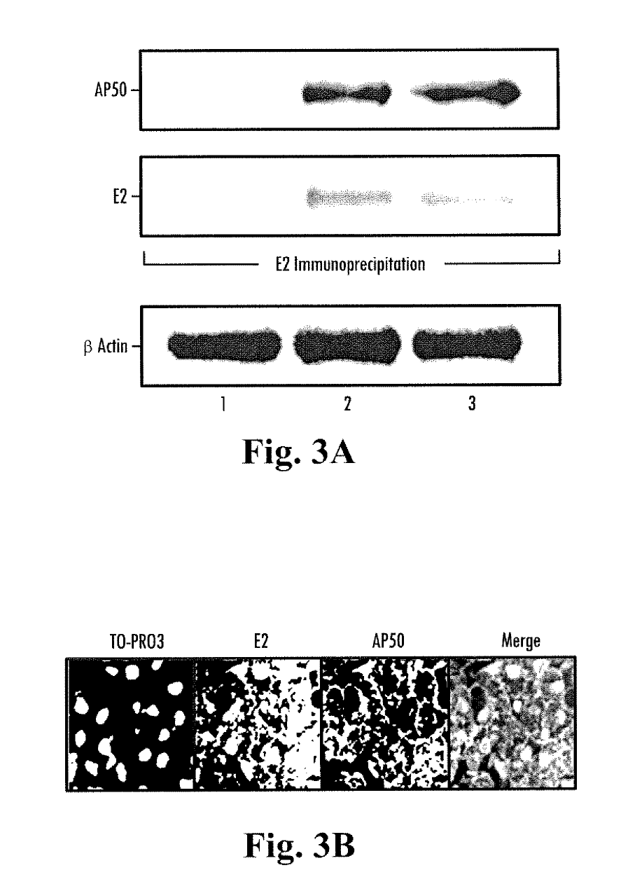

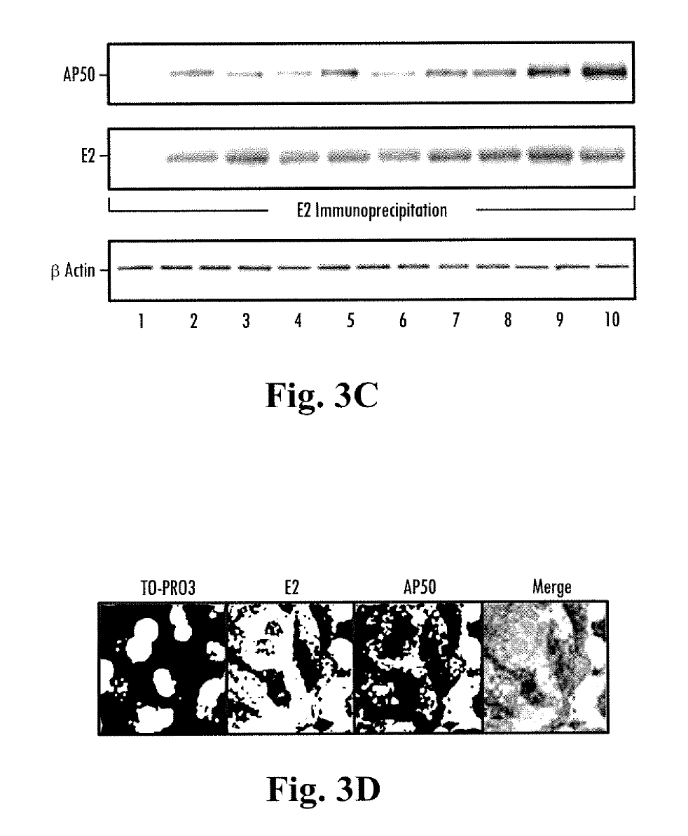

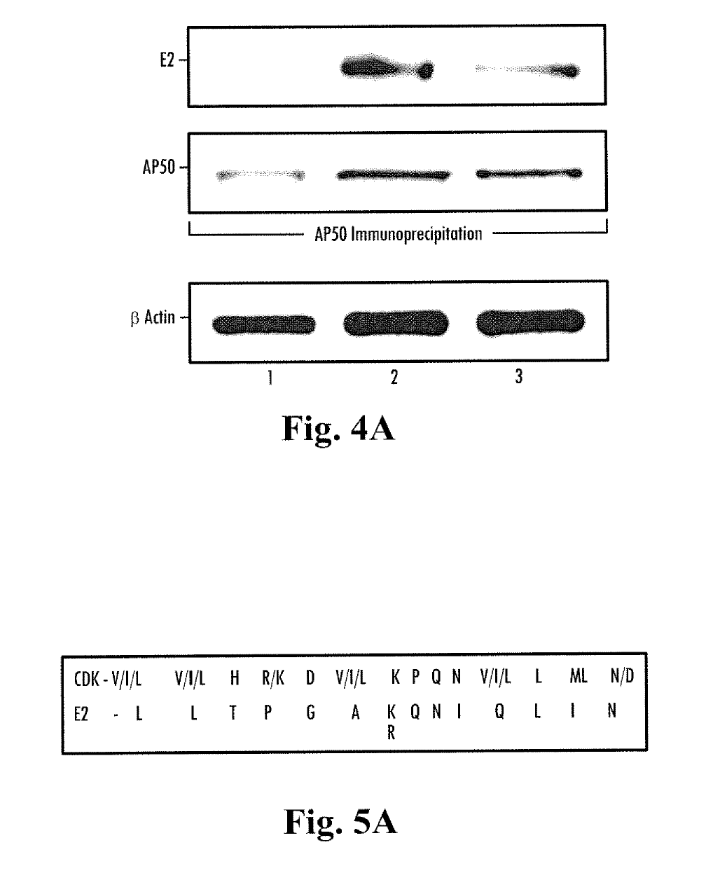

HCV glycoprotein E2 contains a catalytic loop similar to cyclin dependent kinases, associates with cyclin G and shares several motifs and functions with cyclin G associated kinase/auxilin 2(GAK), including a cargo domain and clathrin binding domains. E2 controls endocytosis through phosphorylation of AP-50/μ2, on a target site comprising an amino acid sequence of LIXXQXTG (SEQ ID NO:1), SGREYALKR (SEQ ID NO:32), or LVCLLTPGAKQNIQLI (SEQ ID NO:33), making it a member of the Ark1/Prk1 family of kinases. HCV E2 glycoprotein induces phosphorylation of and associates with the adaptor protein AP-50, a key step for endocytosis, in the liver of HCV infected patients and in HCV-infected cultured human hepatocytes. The association of HCV glycoprotein E2 with AP-50 in HCV-infected livers was determined by co-immunoprecipitation assays (FIGS. 3A and 4), and co-localization by laser-scanning confocal microscopy (FIG. 3B) when compared to uninfected human liver. These data show that the culture system of the invention accurately reflected the interaction between HCV E2 and AP-50 in the liver of HCV-infected patients.

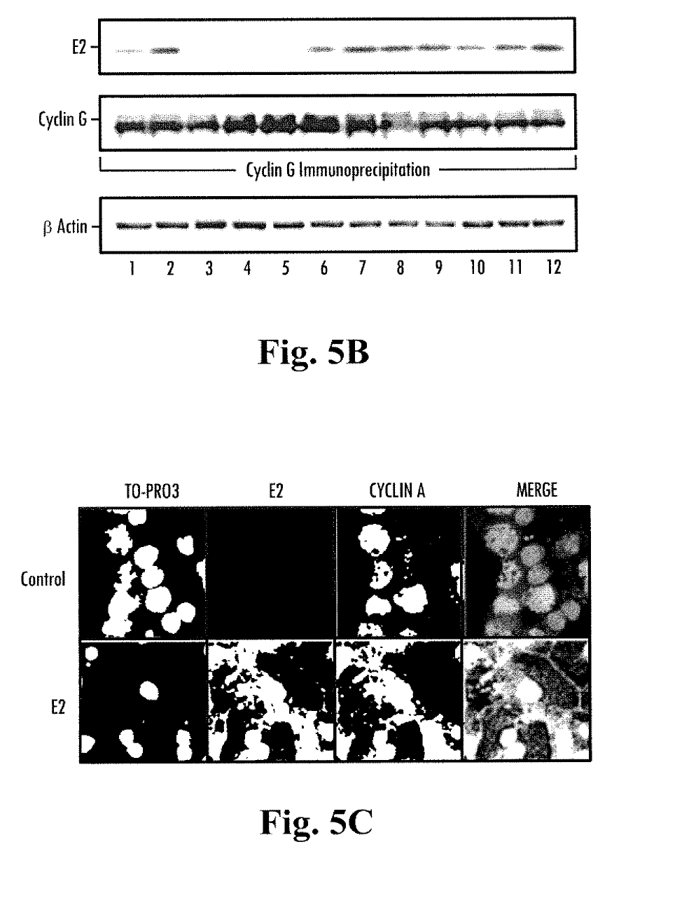

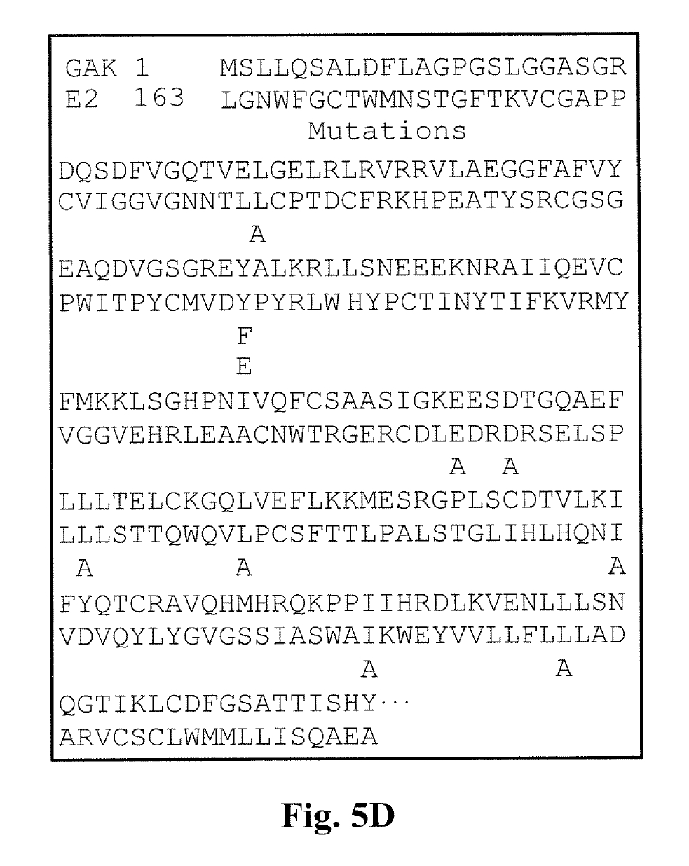

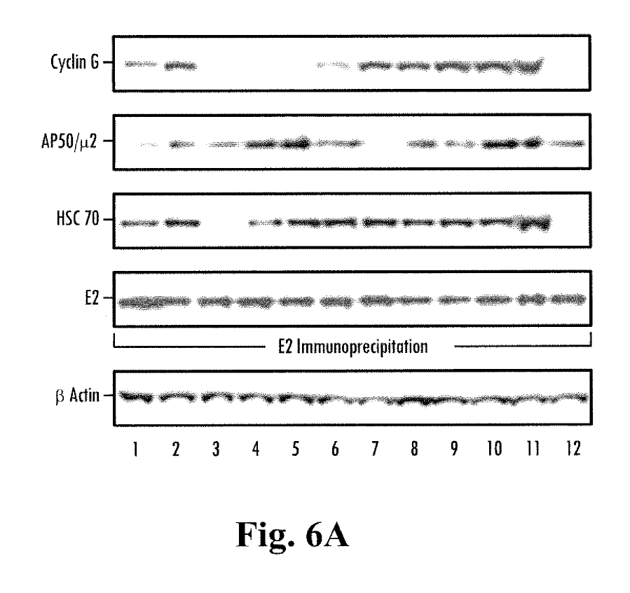

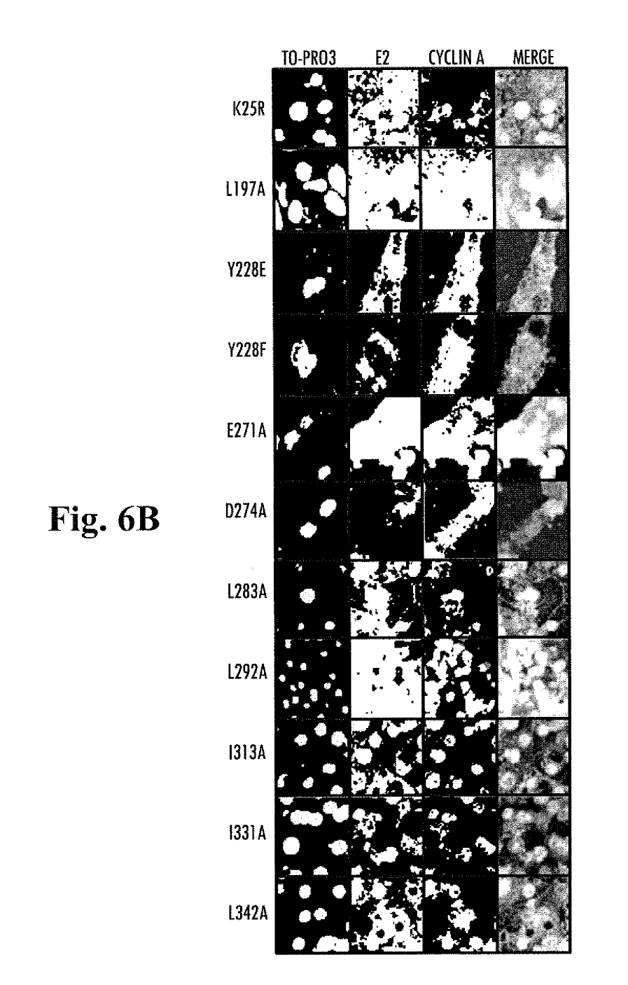

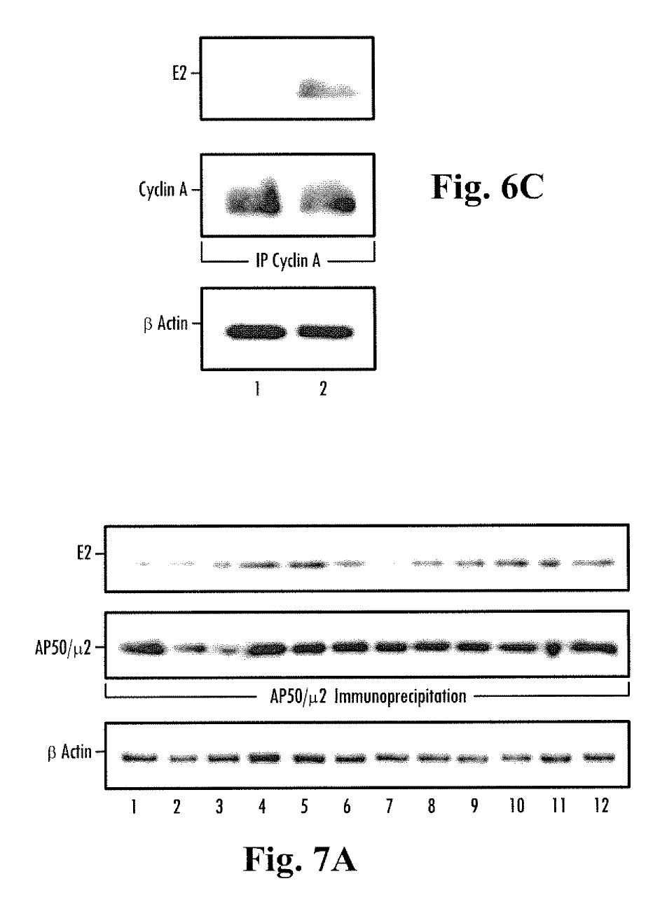

FIG. 5A shows the conserved amino acids of HCV glycoprotein E2 and its 43% homology of this region to cyclic dependent kinases (CDKs), MAP kinases, GSK and Cdc-like kinases (CMGC). The association of E2 with mouse cyclic G is shown in FIG. 5B and FIG. 6A and the association of E2 with its homologous human cyclin A in primary hepatocytes transfected with the recombinant E2 protein is shown in FIG. 5C and FIG. 6B. E2 does not associate with cyclins B, D, E, F, H or T.

Cyclin associated kinase (CAK) hinds to cyclin G, which is also known as auxilin 2 due to its homology to auxilin. FIG. 5D shows that HCV glycoprotein E2 has homology to the kinase region of GAK and several functionally important motifs in E2 are conserved in all of the HCV genotypes and in human GAK. In addition, it has been known that several of the E2 leucines homologous to GAK are indispensable for its association with cyclin C, an L197A mutation of a potential clathrin binding domain (Rodionov et al., J. Biol. Chem. 273, 6005 (1998)), and two mutations Y228E/F of a potential cargo domain (Honing et at, Molecular Cell 18, 519 (2005)) in E2. FIG. 5B and FIGS. 6A and B shows that these mutations disrupt its association to cyclin C. However, the catalytic loop was not indispensable for E2/cyclin G association, as the K25R mutation failed to disrupt this association (FIG. 5B and FIGS. 6A and B). These data support the importance of E2 in hepatitis C viral infection through its ability to control elements of the CME, and induce signal transduction cascades that result in cell survival and proliferation.

In its regulation of receptor endocytosis, GAK was proven to be a kinase that phosphorylates the medium subunits of both AP2, the membrane adaptor complex, and AP1, the trans-Golgi network adaptor complex, AP50/p2 and a1 respectively (Umeda et al., Eur J Cell Biol 79, 336 (2000)). AP2 complexes control CME by providing a bridge between membrane receptor's cargo domains and the clathrin coat. This occurs through binding of the μ2 subunit of AP2 and the clathrin R subunit (Honing et al., Molecular Cell 18, 519 (2005)). This binding has been found to be crucial as clathrin coated pits and TfR endocytosis were inhibited in AP2 depleted cells (Motley et al, The Journal of Cell Biology 162, 909 (2003)). The binding of μ2 to membrane receptors was facilitated by its phosphorylation. In addition, the auxilin homologue of C. elegans was necessary for receptor mediated endocytosis (Greener et at, Nat Cell Biol 3, 215 (2001)) and the Aux1, a yeast homologue, was required for effective vesicle transport (Pishvaee et al., Nat Cell Biol 2, 958 (2000)).

### Example 5

### Mutations of HCV E2 Protein disrupt Its Association with AP50 and HSC70 in the Human Primary Hepatocyte/HCV Infection Culture System

### Mutagenesis

The QuikChange Site-Directed Mutagenesis Kit (Stratagene, Cat.#200519) had been used to mutate amino acids. The QuikChange site-directed mutagenesis method was performed using PfuTurbo DNA polymerase-Stratagene, (cat.# 600250). The oligonucleotide primers were purified by PAGE to reduce the contaminating salts. The template DNAs used for mutagenesis were pIVEX2.6d NS1/E2 and pRSETC NS1/E2. The Full length HCV cDNA was graciously provided by Dr. C. Rice and used to remove the E2 cDNA for the study. Competent E. coli strain with an hsdR17 genotype was used in these studies. Reaction mixtures contained: 10× mutagenesis buffer, 5 μl, template plasmid DNA 5-50 ng, dNTP-mix 300 μM, oligonucleotide primer 1 100-200 ng, oligonucleotide primer 2 100-200 ng, Pfu Turbo DNA polymerase 3U, and H₂O to 50 μL. PCR denaturation, annealing, and polymerization times and temperatures: 1 cycle 30 sec at 98° C., 18 cycles 30 sec at 98° C., 1 min at 55° C. 2 min/kb of plasmid DNA at 68° C., and the last cycle 1 min at 94° C., 1 min at 55° C., 10 min at 72° C. Amplified DNAs were digested by adding 10 units of DpnI directly to the remainder of the amplification reactions and incubated 1 h at 37° C. Competent E. coli were transformed with 1 μl of digested DNA. Plasmid DNA was prepared from 12 independent transformants. DNA preparations were screened for mutations by DNA sequencing, and restriction digestion.

Primers used were;

(SEQ ID NO: 10)

| | |
|---|---|
| NS1/E2 408 | 5′-ACACCAGGCGCCAGGCAGAACATCCAACTG-3′ |
| K/R-S | |
| and | |

(SEQ ID NO: 11) for K25R mutation;

| | |
|---|---|
| 408 K/R-AS | 5′-CAGTTGGATGTTCTGCCTGGCGCCTGGTGT-3′ |

(SEQ ID NO: 12)

| | |
|---|---|
| NS1/E2 L197A-S | 5′-GGCAACAACACCTTGGCATGCCCCACTGAT-3′ |
| and | |

(SEQ ID NO: 13) for L197A mutation;

| | |
|---|---|
| L197A-AS | 5′-ATCAGTGGGGCATGCCAAGGTGTTGTTGCC-3′ |

(SEQ ID NO: 14)

| | |
|---|---|
| NS1/E2 Y228E-S | 5′-TGCATGGTCGACGAGCCGTATAGGCTTTGG-3′ |
| and | |

(SEQ ID NO: 15) for Y228E mutation;

| | |
|---|---|
| Y228/E-AS | 5′-CCAAAGCCTATACGGCTCGTCGACCATGCA-3′ |

(SEQ ID NO: 16)

| | |
|---|---|
| NS1/E2 Y228F- | 5′-TGCATGGTCGACTTCCCGTATAGGCTTTGG-3′ |
| S | |
| and | |

(SEQ ID NO: 17) for Y228F mutation;

| | |
|---|---|
| Y228F-AS | 5′-CAAAGCCTATACGGGAAGTCGACCATGCA-3′ |

(SEQ ID NO: 18)

| | |
|---|---|
| NS1/E2 E271 A- | 5′-CGCTGTGATCTGGCTGACAGGGACAGGTCC-3′ |
| S | |
| and | |

(SEQ ID NO: 19) for E271A mutation;

| | |
|---|---|
| E271A-AS | 5′-GGACCTGTCCCTGTCAGCCAGATCACAGCG-3′ |

(SEQ ID NO: 20)

| | |
|---|---|
| NS1/E2 D274A-S | 5′-CTGGAAGACAGGGCCAGGTCCGAGCTCAGC-3′ |
| and | |

(SEQ ID NO: 21) for D274A mutation;

| | |
|---|---|
| D274A-AS | 5′-GCTGAGCTCGGACCTGGCCCTGTCTTCCAG-3′ |

(SEQ ID NO: 22)

| | |
|---|---|
| NS1/E2 666 LA- | 5′-CTCAGCCCGTTAGCACTGACCACTACACAG-3′ |
| S | |
| and | |

(SEQ ID NO: 23) for L283A mutation;

| | |
|---|---|
| 666 L/A-AS | 5′-CTGTGTAGTGGTCAGTGCTAACGGGCTGAG-3′ |

(SEQ ID NO: 24)

| | |
|---|---|
| NS1/E2 675 | 5′-ACACAGTGGCAGGTCGCACCGTGTTCCTTC-3′ |
| L/A-S | |
| and | |

(SEQ ID NO: 25) for L292A mutation;

| | |
|---|---|
| 675 L/A-AS | 5′-GAAGGAACACGGTGCGACCTGCCACTGTGT-3′ |

(SEQ ID NO: 26)

| | |
|---|---|
| NS1/E2 696 | 5′-CACCTCCACCAGAACGCAGTGGACGTCCAG-3′ |
| I/A-S | |
| and | |

(SEQ ID NO: 27) for 1313A mutation;

| | |
|---|---|
| 696 1/A-AS | 5′-CTGCACCTCCACTGCGTTCTGGTGGAGGTG-3′ |

(SEQ ID NO: 28)

| | |
|---|---|
| NS1/E2 714 | 5′-GCGTCCTGGGCCGCAAAGTGGGAGTACGTC-3′ |
| I/A-S | |
| and | |

(SEQ ID NO: 29) for 133IA mutation;

| | |
|---|---|
| 714 I/A-AS | 5′-GACGTACTCCCACTTTGCGGCCCACGACGC-3′ |

(SEQ ID NO: 30)

| | |
|---|---|
| NS1/E2 725 | 5′-GTTCTCCTGTTCCTTGCACTTGCAGACGCG-3′ |
| L/A-S | |
| and | |

(SEQ ID NO: 31) for L342A mutation

| | |
|---|---|
| 725 L/A-AS | 5′-CGCGTCTGCAAGTGCAAGGAACAGGAGAAC-3′ |

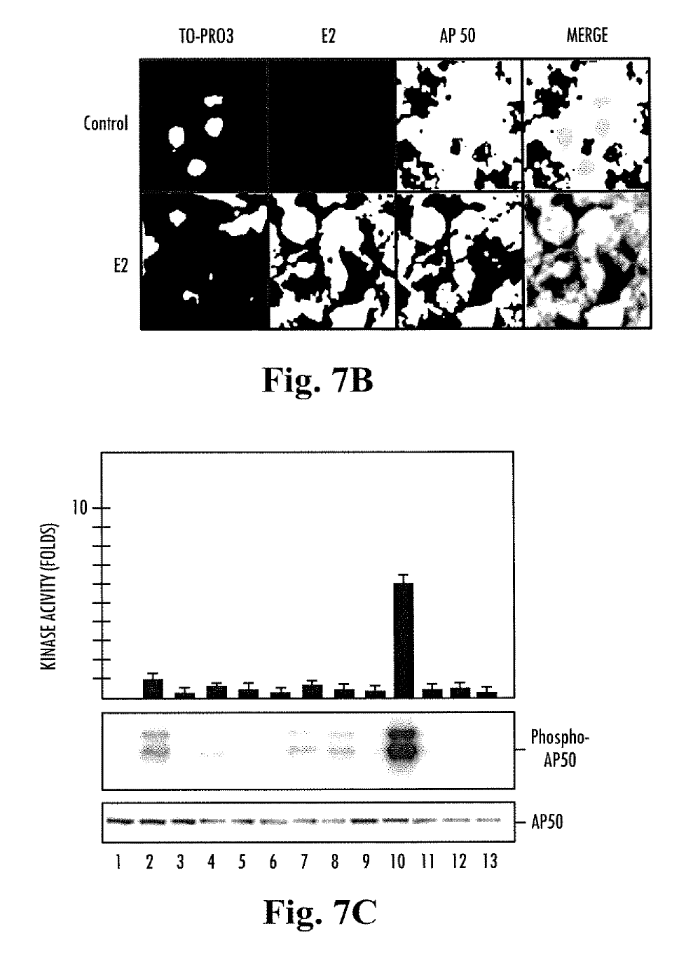

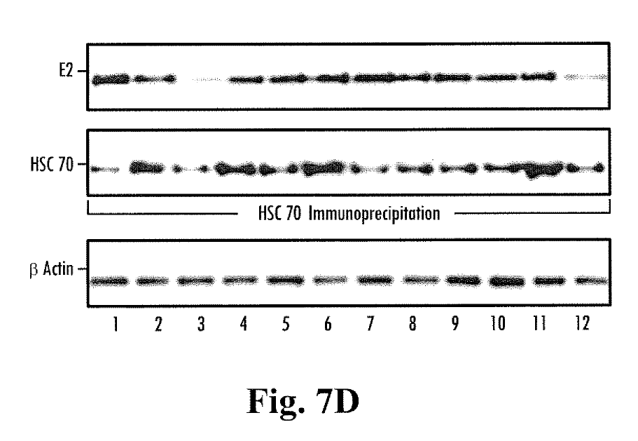

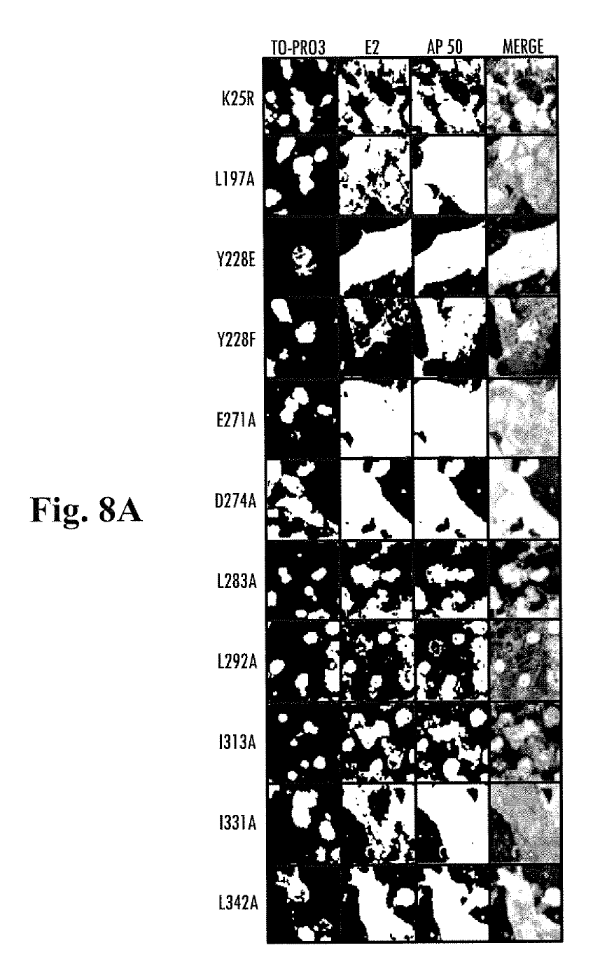

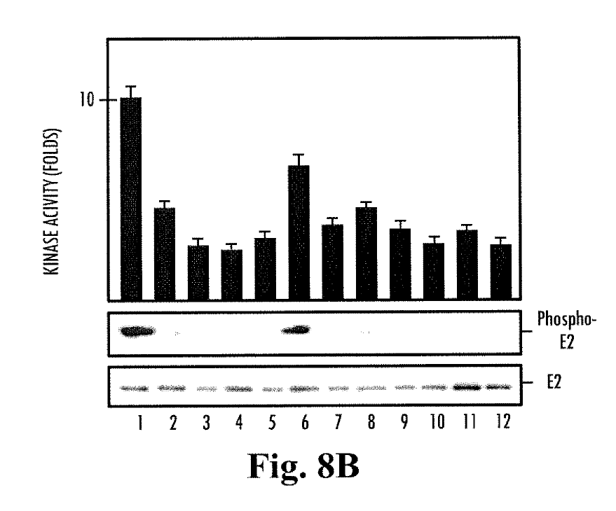

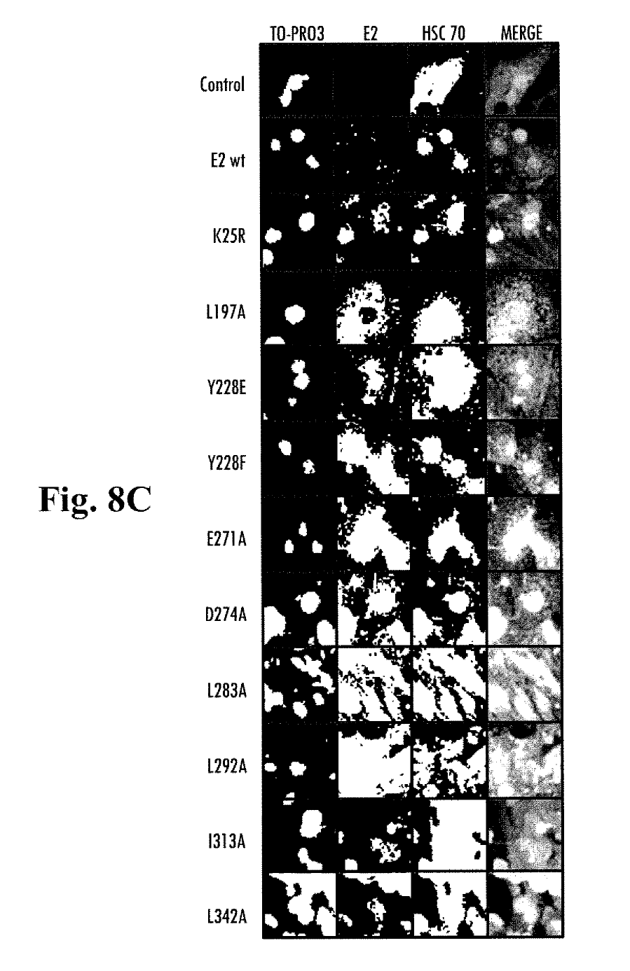

HCV E2 in cell transfections was able to associate with AP50/p2 and recombinant E2 could phosphorylate AP50/p2 on threonine 156 (FIG. 7C), the same residue phosphorylated by GAK, and like most kinases, could also self-activate through autophosphorylation (FIG. 8B). Although few mutations of E2 disrupt its association with AP50/p2 (FIG. 7A, B and FIG. 8A), mutations of K25R in the kinase catalytic loop and L197A in the clathrin binding domain, the two Y228E/F in the cargo domain, and the E271A, D274A, L283A, I313A, I331A and L342A in the potential plasma membrane/endosome signal sequences all decreased the phosphorylation of AP50/p2 by E2 (FIGS. 7A and C). As part of the endocytic vesicles, E2 was also able to associate with HSC70 (FIG. 7D and FIG. 5C). The association of GAK with HSC70 was known to occur through its J domain (Zhang et al., Traffic 6,1103 (2005)). However, Y228E and L342A mutations in the cargo domain and PM/endosome signal sequences disrupted the E2/HSC70 associations (FIG. 7D and FIGS. 8A and 8C).

### Example 6

### HCV E2 Protein Increased Clathrin (HC) Expression and Endocytosis of Transferrin (Tf) in the Human Primary Hepatocyte/HCV Infection Culture System

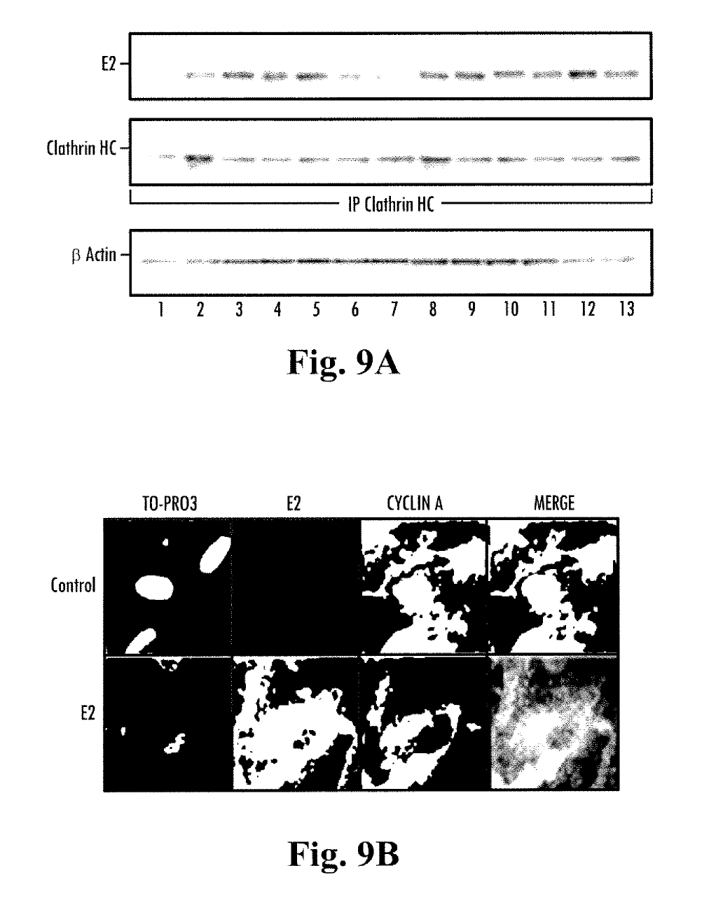

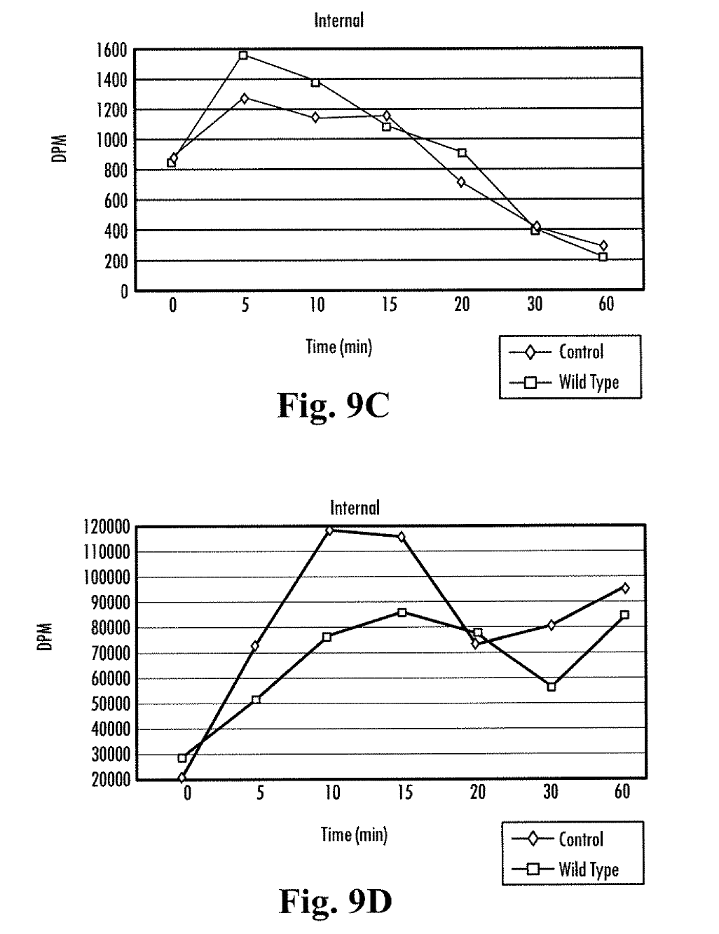

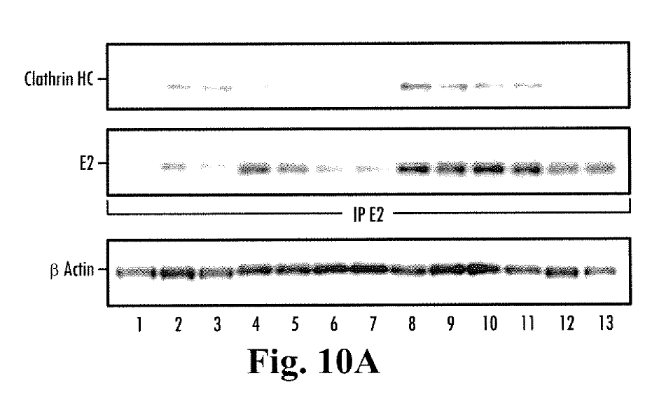

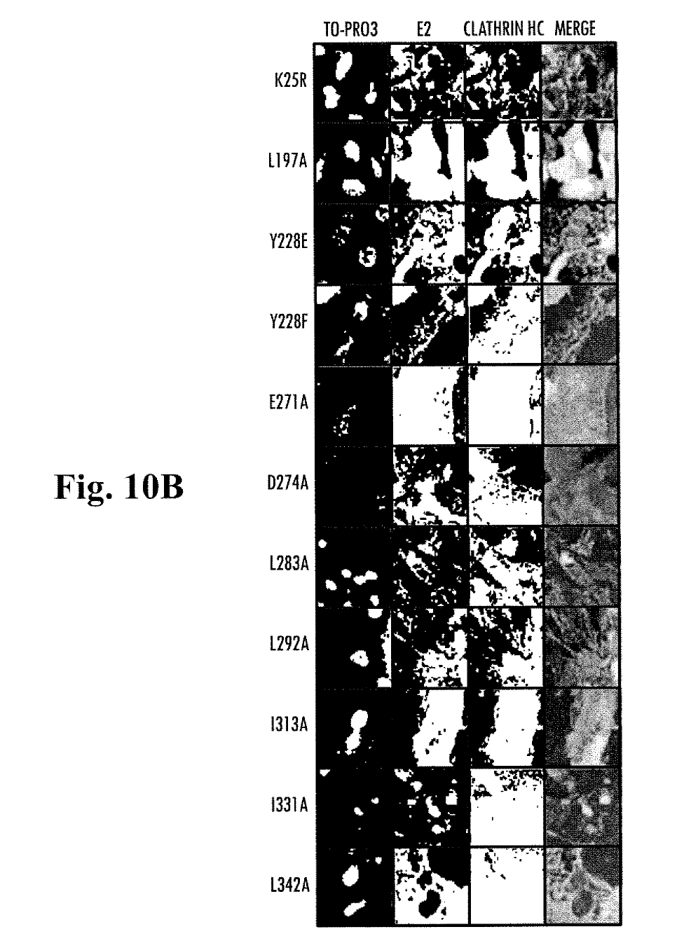

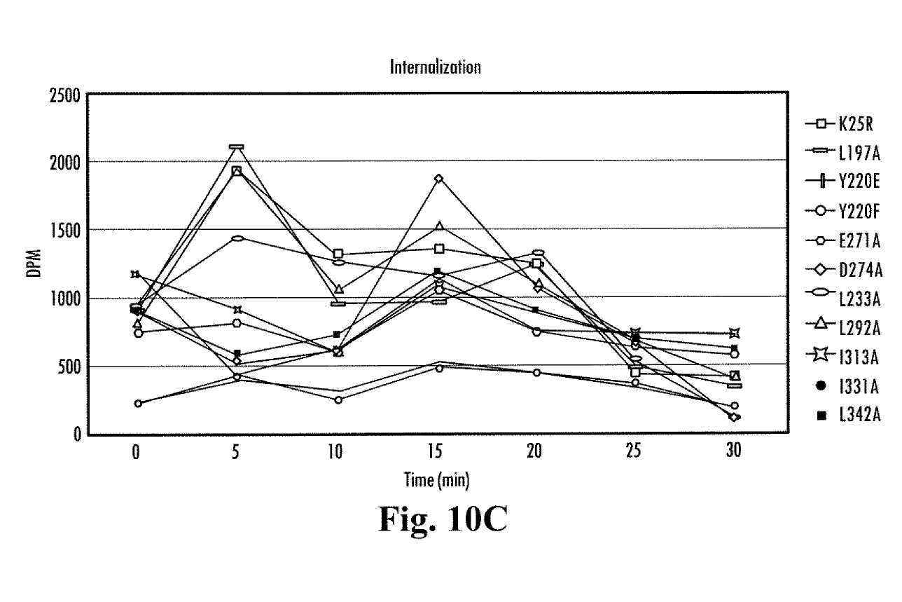

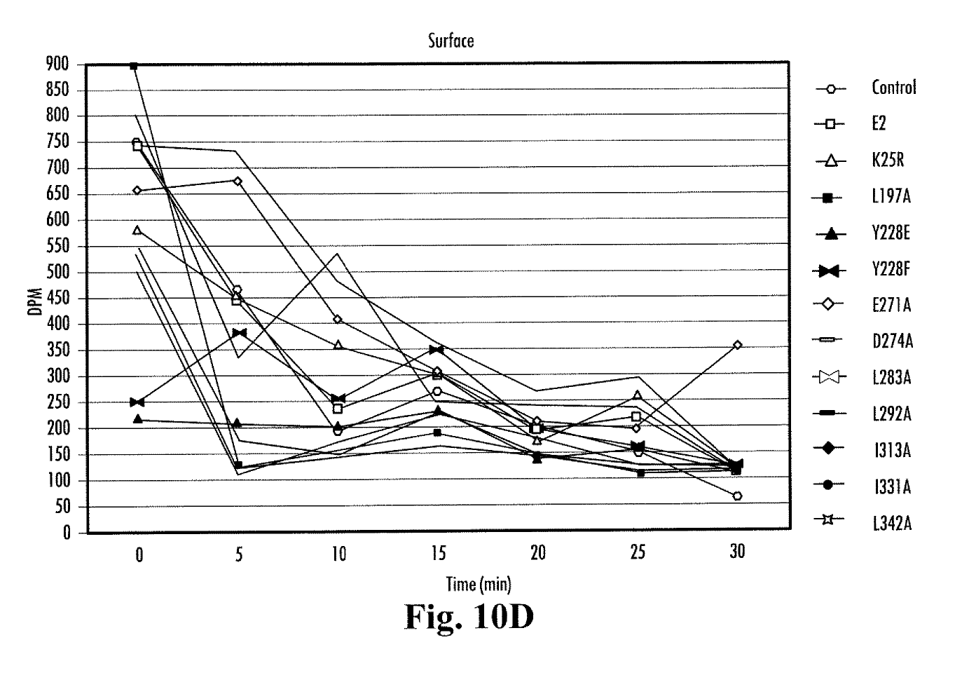

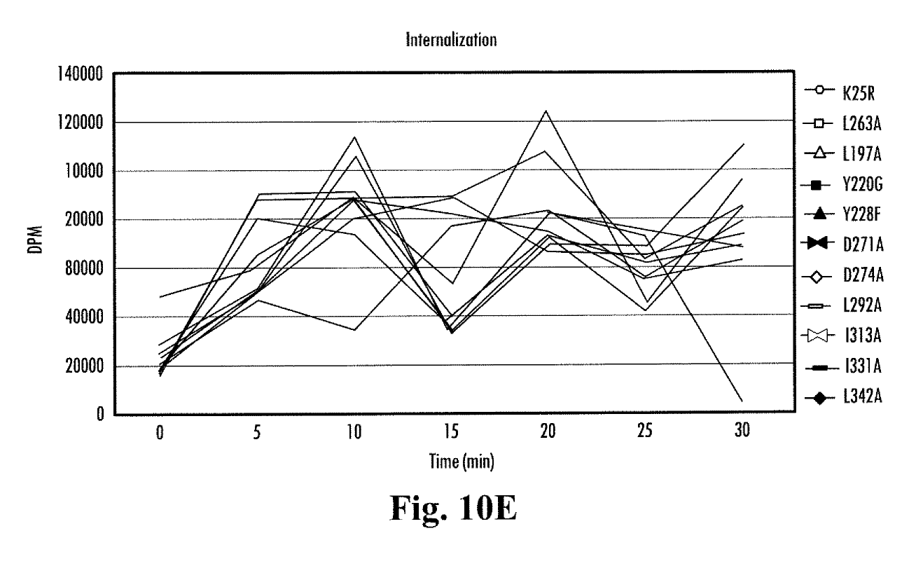

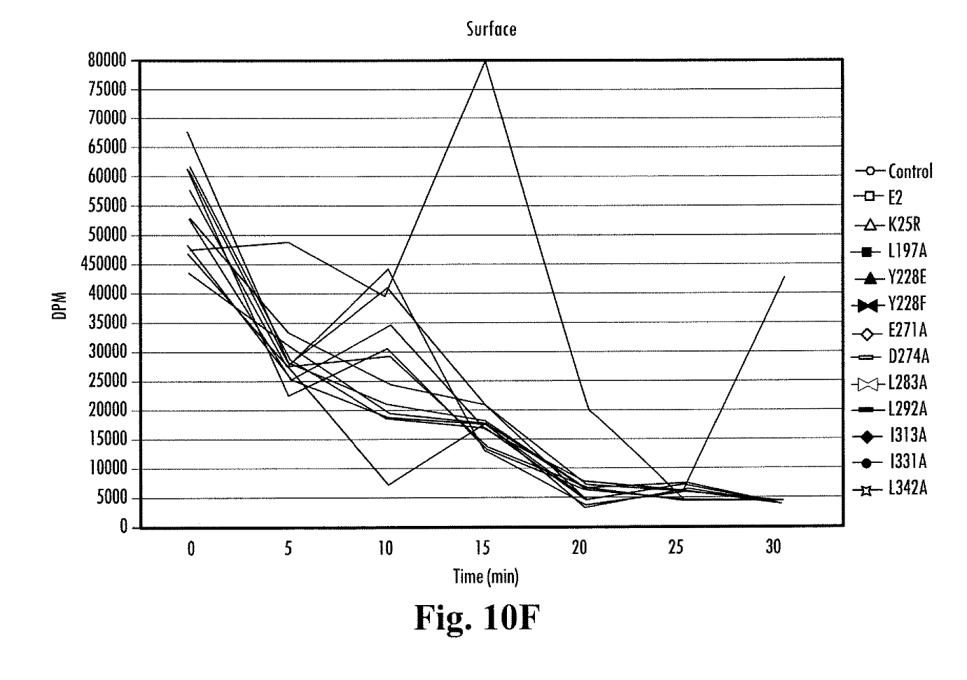

Clathrin heavy chain (HC) has been shown to be indispensable for endocytosis and cell survival. The study had reported in DT40 lymphocytes that when clathrin HC was eliminated through homologous recombination, there was no endocytosis of avian leukosis virus and the cells died from a decrease in phosphorylation of Akt, leading to apoptosis (Wettey et al., Science 297, 1521 (2002)). HCV E2 was able to associate with the clathrin HC as part of the endocytic vesicles, increased the expression of clathrin HC (FIG. 9 and FIGS. 10A and B), and moved the majority of the clathrin from the PM into the cytoplasm, presumably increasing endocytosis (FIG. 9B). Mutations of L197A in the clathrin binding domain, Y228E/F in the cargo domain, and I331A and L342A in the PM/endosomal signal sequences of E2 abolished both the increase in clathrin HC expression (FIG. 9A and FIG. 10A) and the increase in clathrin endocytosis (FIG. 10D).

The phosphorylation of AP50A2 is critical for transferrin receptor endocytosis (Motley et al, The Journal of Cell Biology 162, 909 (2003)). Extracellular iron circulates in plasma bound to transferrin (Tf), nonreactive and in the hepatocytes is internalized through transferring receptor-2 (TfR2), clathrin-coated pits regulated endocytosis (Hentze et al, Cell 117, 285 (2004)). In GAK transiently depleted Hela cells, the internalization of transferrin receptor trafficking is markedly decreased (Lee et al., J Cell Sci 118, 4311 (2005)).

GAK was found to be involved in both CME and transgolgi network (TGN) trafficking, with its kinase activity being indispensable for Tf uptake (Zhang et al., Traffic 6, 1103 (2005)). The HCV E2 protein also increased the internalization of Tf in primary hepatocytes. In primary hepatocytes transfected with the E2 protein and given ¹²⁵I-Tf, the internalization of Tf was faster and greater than in control hepatocytes not given the E2 protein (FIG. 9C). Since the amount of total surface-bound Tf remained unchanged (FIG. 10D), this increased Tf uptake reflected an induction of early endocytosis. Several of the E2 mutants failed to internalize Tf efficiently (FIG. 10C). The Y228E/F cargo domain mutations dramatically reduced the internalization of Tf (FIG. 10C), due to an inability to attach to the cell surface (FIG. 10D). Several of the mutations, including E271 A, D274A, and I313A, within the PM/endosomal and L342 within the clathrin binding signal sequence of E2 had decreased internalization (FIGS. 10C and D). This disruption of Tf internalization due to mutated E2 that led to decreased binding of Tf at the cell surface was possibly due to a failure to present Tf R2 for the binding of Tf, because of an inability to associate with either clathrin or AP50/μ2 in the endocytic vesicle and causing a blockade of the CME.

### Example 7

### HCV E2 Protein Decreased Internalization of Epidermal Growth Factor Receptor (EGFR) in the Human Primary Hepatocyte/HCV Infection Culture System

### Transferrin and EGF Endocytosis

Radioisotopes were purchased from Perkin Elmer. Transferrin (human) [¹²⁵I]-diferric (Cat#NEX212) and Epidermal Growth Factor (murine) [¹²⁵I] (Cat#NEX160). Plate was removed from incubator and put in cold room. 1 μci of ¹²⁵I was immediately added to each well and left in cold room for exactly 30 minutes. ¹²⁵I was removed by washing 2× with PBS. 2 ml/well DME High Glucose was added (Gibco) and cells were incubated at 37¹ C for indicated time points. At each time point media was removed and 500 μl of surface bound buffer added (0.5% acetic acid, 0.5M NaCl, in PBS) for 2 minutes at room temperature. Surface bound buffer was removed and put into corresponding and saved for counting as this is the surface bound fraction. Cells were washed with 1× PBS and 500 μl of internal buffer (1% Triton X-100+0.5% SDS in PBS) was added and incubated at 37° C. for 5 minutes. Cells were harvested and radioactivity was determined using a Beckman LS6500 liquid scintillation counter with 5 ml Bio-Safe II counting cocktail.

EGFR signaling has also been shown to be regulated by GAK through its control of CME (Vieira et al, Science 274, 2086 (1996); Zhang et al., PNAS 101, 10296 (2004)). In cells expressing a mutant form of dynamin, causing a conditional and specific defect in receptor-mediated endocytosis early EGF-dependent cell proliferation was enhanced, but endocytic trafficking was required for downstream activity of MAP kinases (Vieira et al., Science 274, 2086 (1996); Holgado-Madruga et al, Nature 379, 560 (2006); Marshall, Cell 80, 179 (1995)). GAK is believed to act upon cellular trafficking subsequent to dynamin regulation (Huang er at, J. Biol. Chem. 278, 43411 (2003)).

GAK has also been shown to be responsible for controlling EGFR expression, activation, and downstream signaling. In GAK stably selected knock-down cells, through small hairpin RNAs, EGF expression, internalization, and downstream signaling was increased (Zhang et al., PNAS 101 10296 (2004)) suggesting that GAK down-regulates EGF activity and its downstream signal transduction cascade. Using protein transfected primary mouse hepatocytes, the internalization of EGFR was decreased by HCV E2 (FIG. 9D). The Y228F mutant of the tyrosine in the cargo domain, has a greatly delayed, though almost normal ¹²⁵I internalization of EGF, possibly due to its delayed but increased binding of EGF at the PM (FIGS. 10E and F). This decreased internalization of EGF, below that of wild type E2, is possibly due to a failure to present EGFR at the cell surface or incorporate EGFR into endocytic vesicles. The K25R mutant of the CMGC catalytic loop is one of the most diminished in its capacity to internalize EGF, possibly due to its inability to phosphorylate AP50/N2 and connect the cell membrane signals to the clathrin_{R}subunit. Tyrosine phosphorylation of the 02 subunit of AP2 is required for the recruitment of EGFR into coated pits and it has been suggested that 02 phosphorylation is mediated by the receptor interaction with the AP50/μ2 subunit of AP2 (Huang et al., J. Biol. Chem., 278, 43411 (2003)).

### Example 8

### HCV E2 Induced Primary Hepatocyte Proliferation Through Activation of the Phosphotidylinositol-3 (PI-3) Kinase Cascade in the Absence of External Growth Stimuli

### Mouse Primary Hepatocyte Cultures

Hepatocytes were isolated by a modified perfusion technique introduced by Seglen (P. O, Seglen, Methods Cell Biol. 13, 29 (1976)). A liver with calcium-free HBSS supplemented with calcium-free HBSS supplemented with 0.5 mM EGTA for 20 to 30 min and then with 0.05% collagenase [Sigma] dissolved in L-15 medium (with calcium) at 37° C. until the tissue was fully digested. The digested liver was removed, immediately cooled with ice-cold L-15 medium and the cell suspension was strained through serial progressively smaller stainless steel sieves, with a final filtration through 100-micron and 60-micron nylon mesh. The filtered cell suspension was aliquoted into 250-ml tubes and centrifuged three times at 40 g for 3 min at 4° C.

Cells were re-suspended in Hepatocyte Plating Media (500 ml DMEM high glucose; 20% FBS) and plated at a concentration of at 0.625 10⁶ cells/mL. Diluted collagen (type 1, rat tail-BD Cat. #354236) (50 ug/ml in 0.02N acetic acid) was used for coating coverslips and plates in about 10 ml (enough to cover them) at room temperature for one hour. The collagen solution was then removed and rinsed once with PBS. After the cells attached (<18 hrs), the HPM was replaced by Hepatocyte Media (500 mL DMEM high glucose; 30 mg L-methionine; 104 mg L-leucine; 33.72 mg L-ornithine; 200 μL of 5 mM stock dexamethasone; 3 mg Insulin).

### ³H Thymidine Incorporation

Cells were transfected with Chariot (Active Motif cat #30100). Transfection reagent was removed and 2 ml/well media was added and incubated at 37° C. for 2 hours. Either EGF (upstate cat #01-101) at 25 ng/ml or TGFa (EMD cat. #PF008) at 25 ng/ml were added. 1 μci/ml Thymidine, [methyl-³H] (Perkin Elmer Cat #NET027Z) was added to cells and they were incubated at 37° C. for 48 hours. Media was removed and the cells were washed 2× with ice cold PBS. 0.5 ml of cold 10% Trichloroacetic acid (TCA) was added and incubated at room temperature for 1 hour. TCA was removed and cells were rinsed with ethanol. Cells were harvested in 0.5 ml of 0.1 M NaOH containing 1% SDS. Radioactivity was determined using a Beckman LS6500 liquid scintillation counter.

Phosphotidylinositol 4,5-biphosphate (PIP2) is required for clathrin-mediated endocytosis (Paolo et al., Nature 431, 415 (2004); M. R. Wenk et al., PNAS 101, 8262 (2004)). PIP2 is a phospholipid making up 1% of the cytoplasmic leaflet of the plasma membrane (McLaughlin et al., Nature 438, 605 (2005)). The AP2 complex is recruited exclusively to PIP2 anchored in the plasma membrane where AP2, through its AP50/μ2 subunit, when phosphorylated, binds to the cargo domains of receptors and incorporates them into the clathrin-coated endocytic vesicles. It has been reported that AP2 binding to the cargo domains of receptors and acidic dileucine clathrin motifs is contingent upon recognition of PIP2 (Honing et al, Molecular Cell 18, 519 (2005)) and AP2 binds PIP2 through it's α and μ2 subunits (Rohde et al., The Journal of Cell Biology 158, 209 (2002)).

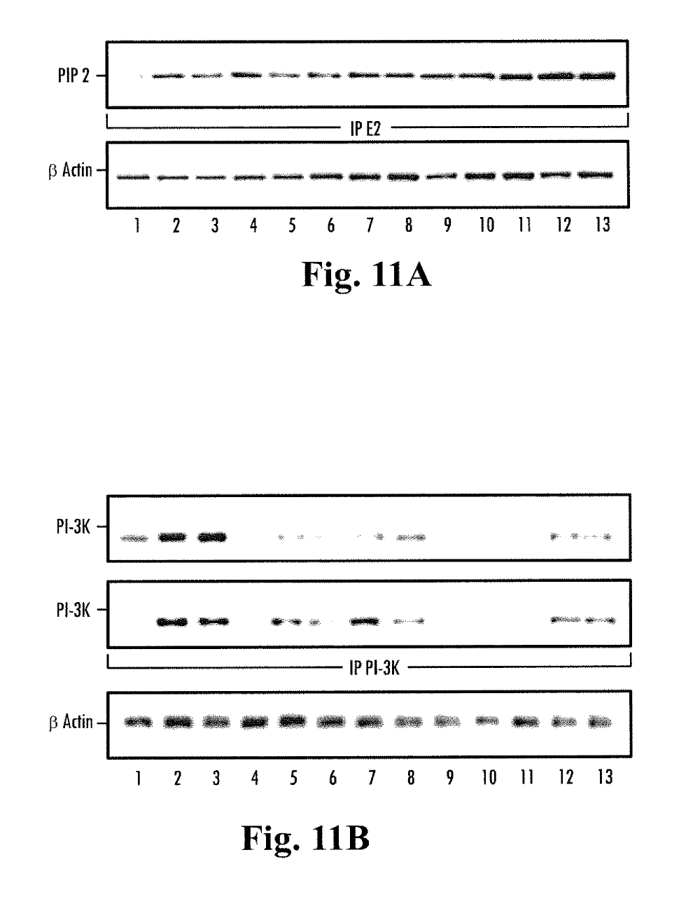

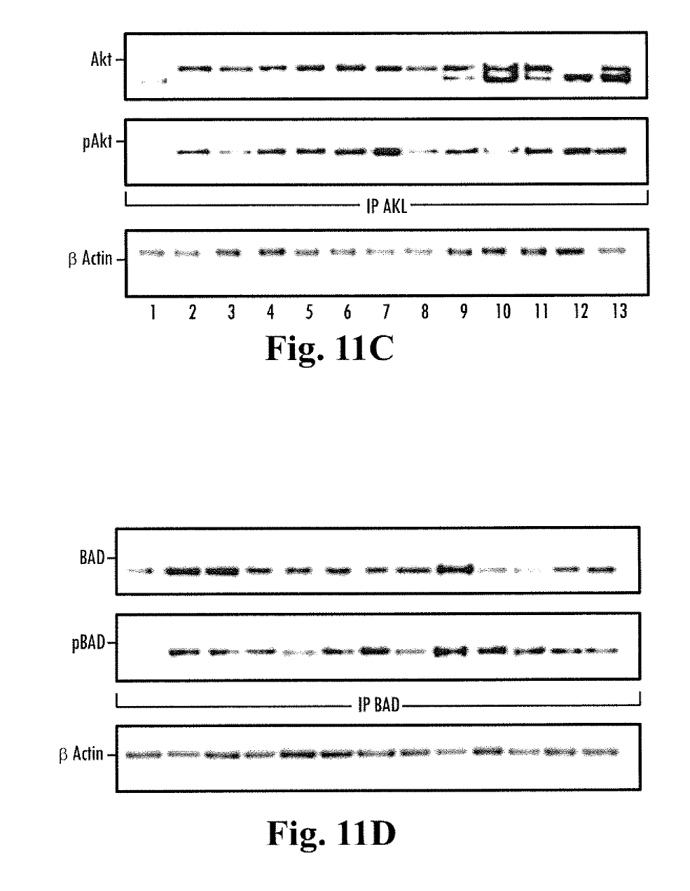

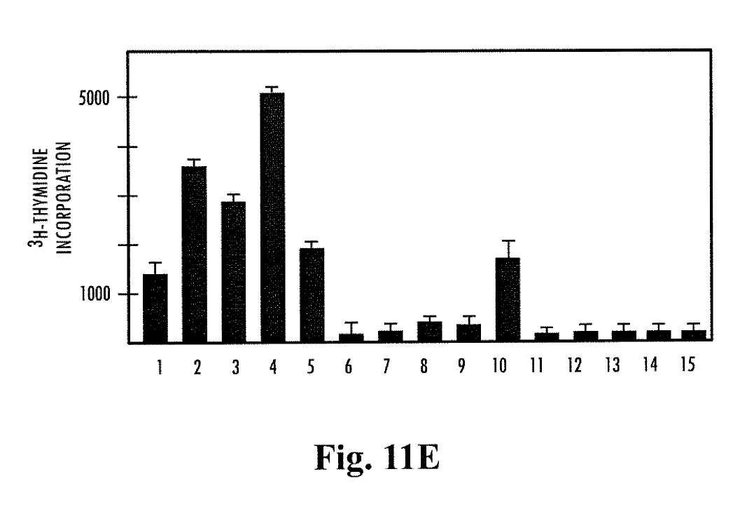

HCV E2 protein transfected into mouse hepatocytes caused an increase in PIP2 (FIG. 11A), which could contribute to the increased endocytosis of these cells. Phophoinositol-3 kinases (PI-3K), principally a p110 catalytic subunit, becomes activated, usually through growth factor stimulation and converts PIP2 to phosphoinositol-3,4,5-triphopshate (PIP3). Signaling proteins with membrane binding pleckstrin-homology domains (PH), Akt and phosphoinositol dependent kinase 1 (PDK1) are recruited to activated PI3K, and activated PDK1 is able to activate Akt through phosphorylation. Activated Akt phosphorylates a multitude of proteins that affect cell growth, cell cycle entry, and cell survival.

Akt phosphorylates BAD, preventing its association with Bcl-2 and Bcl-XL, blocking apoptosis. PDK1 phosphorylates and activates other protein kinases, including p70 S6-kinase which activates the translation of cell growth genes (Cantley, Science 296, 1655 (2002)). E2 not only increased PIP2, but also PI3K, PDK1 and Akt, and their activities (FIGS. 11B, C and D), in the absence of extracellular growth factors. BAD was phosphorylated in cells given E2 (FIG. 11E), HCV E2 not only blocked apoptosis through the activation of this signal transduction cascade, but induced cell proliferation as measured by DNA replication through [³H] thymidine incorporation, above that of known oncogenic stimuli, EGF and TCFα (FIG. 11F).

### Example 9

### The Dominant Negative AP-50 Peptide

Peptide Synthesis

The dominant negative peptide was synthesized by Celtek Biosciences, LLC, to greater than 95% purity.

### Expression of Recombinant Proteins

For expression of pIVEX2.6d NS1/E2 recombinants the cell-free protein expression system for in vitro transcription/translation, Rapid Translation System (RTS) 500 ProteoMaster E. coli HY Kit (Roche, Cat#3 335 461) and RTS ProteoMaster Instrument (Roche) was used.

### Purification of Recombinant Proteins

Immunoprecipitation of the HA-tagged protein NS1-E2 was done using Anti-HA Affinity Matrix (Roche, cat.# 1 815 016) following the Roche protocol. Purified samples are analysed by Western Blotting with Coat Anti HCVE2 antibody, (Biodesign, Cat.# B6558G)

### Recombinant Protein Transfection

100 μl of 25% DMSO were combined with 6 μl of Chariot (Active Motif). In a separate tube, 2 μg of recombinant protein was brought up to 100 μl with PBS. The Chariot solution and the protein dilution were combined and let incubate for 30 minutes at room temperature. The media was removed from plated hepatocytes and the 200 μl of Chariot-protein complex was added. 400 μl of hepatocyte media was added and incubation was continued for 1 hour at 37° C. 1 ml hepatocyte media was then added and Incubation continued at 37° C. for two hours. The Chariot-protein complex was removed and replaced with hepatocyte media overnight.

### Immunostaining and Confocal Microscopy

Primary hepatocytes were fixed with 50/50 acetone/methanol at −20° C. for 20 minutes and allowed to air dry at room temperature. Blocking of non-specific epitopes was done with PBS+3% BSA for 30 minutes at room temperature. Primary antibodies were used at 1:100 dilutions in PBS+3% BSA, for 1 hour at room temperature washes were done with PBS. Secondary antibodies were made in chicken and conjugated to either Alexa 488 or 594 (Molecular Probes) and used at 1:100 dilutions in PBS+3% BSA for 30 minutes at room temperature, Fluorescent labels were observed using a confocal microscope. At least 100 cells were analyzed per experimental point. We analyzed the nuclear morphology by staining cells with TO-PRO-3 (Molecular Probes). Primary antibodies used were to cyclin A, HSC 70 (Santa Cruz Biotechnology), AP50, clathrin HC (BD Transduction Labs), and HCV E2 (Biodesign).

### Immunoprecipitation and Western Blot Analysis

Cells were harvested and lysed with Nonidet P-40 lysis buffer (50 mM Tris-HCl, pH 7.5/150 mM NaCl/1% Nonidet P-40/5 pg of leupeptin per ml/5 pg of pepstatin per ml/0.5 mM phenylmethylsulfonyl fluoride/1 mM sodium fluoride/100 pM sodium vanadate/10 mM R-glycerol phosphate) for 10 min on ice. Extracts were cleared of cell debris by centrifugation at 10,000×g for 10 min in a microcentrifuge at 4° C. 500 kg of protein lysate from each were immunoprecipitated with 2 μg of primary antibody for 2 h and then 45 pl of protein A/G+ agarose beads for 45 min. The beads were washed three times with Nonidet P-40 lysis buffer. For Western blotting, the immunoprecipitates or protein lysates were boiled with one third volume of 3× SDS buffer (150 mM Tris-HCl, pH 6.8/300 mM DTT/6% SDS/0.3% Bromophenol blue/30% glycerol) and separated on a 7.5% or 10% SDS polyacrylamide gel, followed by transfer to a poly(vinylidene difluoride) membrane. The blots were then blocked with 5% dry milk or 2% gelatin (for 4G10) and incubated with primary antibodies. Primary antibodies used were to cyclin C, HSC 70(Santa Cruz Biotechnology), AP50, clathrin HC (BD Transduction Labs), and HCV E2 (Biodesign). Incubations were overnight or 45 min., washed with Tris-buffered saline-Tween (150 mM NaCl/10 mM Tris-HCl/0.2% Tween 20), then incubated with horseradish peroxidase-conjugated anti-rabbit or anti-mouse secondary antibodies (Amersham Biosciences) for 30 min, washed again, and developed by enhanced chemiluminescence (Amersham Biosciences). The signals were visualized by exposure to Kodak X-Omat blue XB-1 film.

### Kinase Activity Assays

AP50 was immunopurified from untransfected primary mouse hepatocytes and subjected to heat inactivation of any associated kinases. Recombinant wild type or mutated E2 was combined with AP50 in the presence of ³²P ATP (MP Biomedicals cat.#35020) and kinase buffer (50 mM Tris-HCL, pH 7.5, 5 mM MgCl2). The reaction was incubated at room temperature for 1 hour, and run on an SDSPAGE, transferred to a membrane and exposed to film overnight and analyzed on a Kodak 4000MM Imaging Station.

Determination of Cell Toxicity

Toxicity of AP-50 peptides to human hepatocyte cultures was determined by measuring lactic dehydrogenase (Sigma) in the medium.

Statistical Analysis

Results are expressed as mean (±SEM) of at least triplicates unless stated otherwise. Either the Student-t or the Fisher's exact test was used to evaluate the differences of the means between groups, with a P value of <0.05 as significant.

As discussed above, several of the mutants are unable to induce proliferation, notably K25R in the kinase catalytic loop, Y228E/F in the cargo domain and most of the mutants in the PM/endosome signal sequences. This suggests that all of these motifs in E2 are necessary for this increase in cellular proliferation. Therefore, an induction of endocytosis, in the absence of growth factors can stimulate abnormal proliferation and that a blockade of E2 with a dominant negative AP50 peptide would inhibit HCV infection.

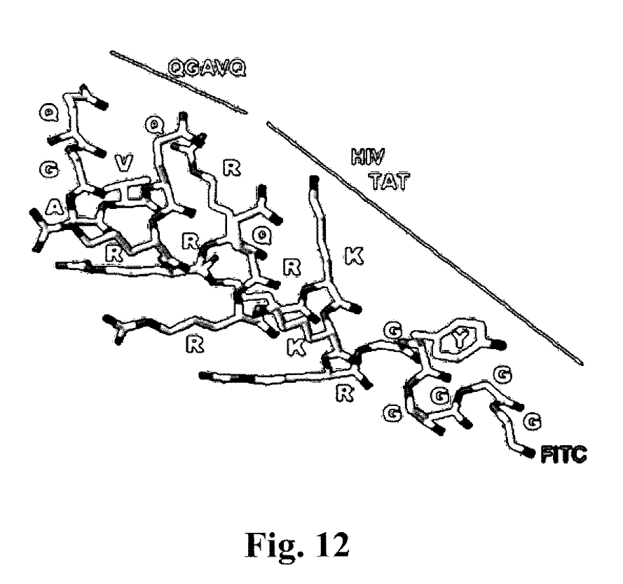

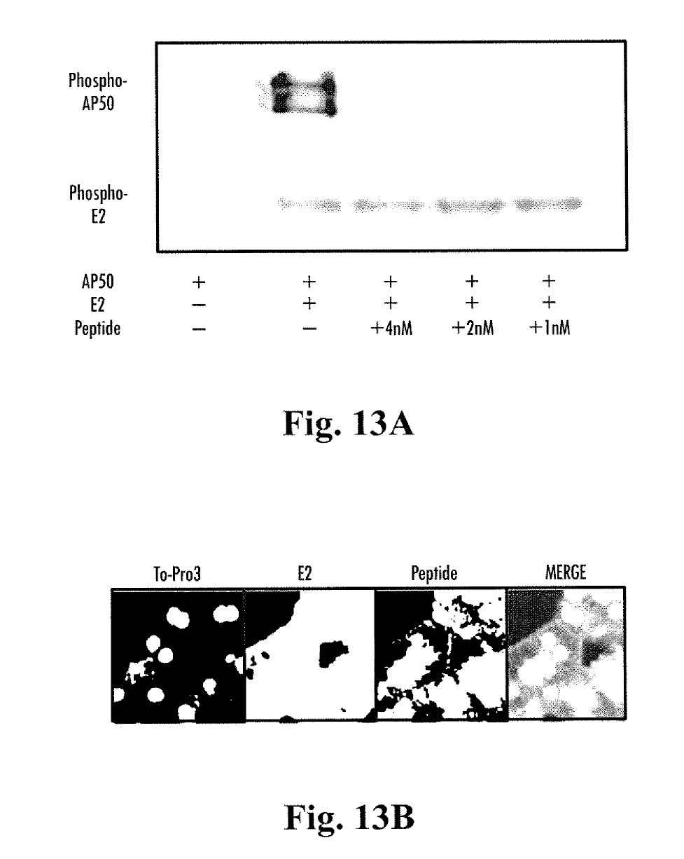

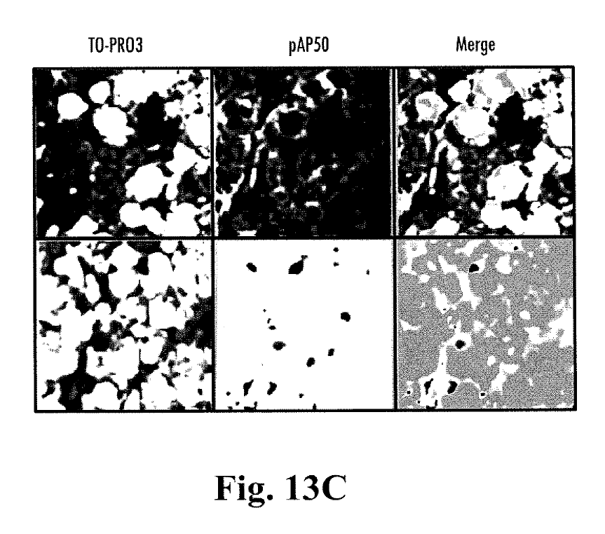

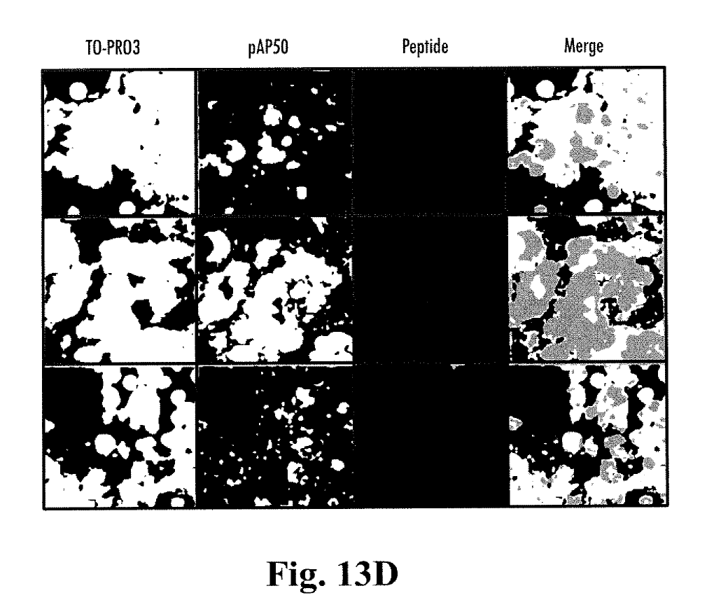

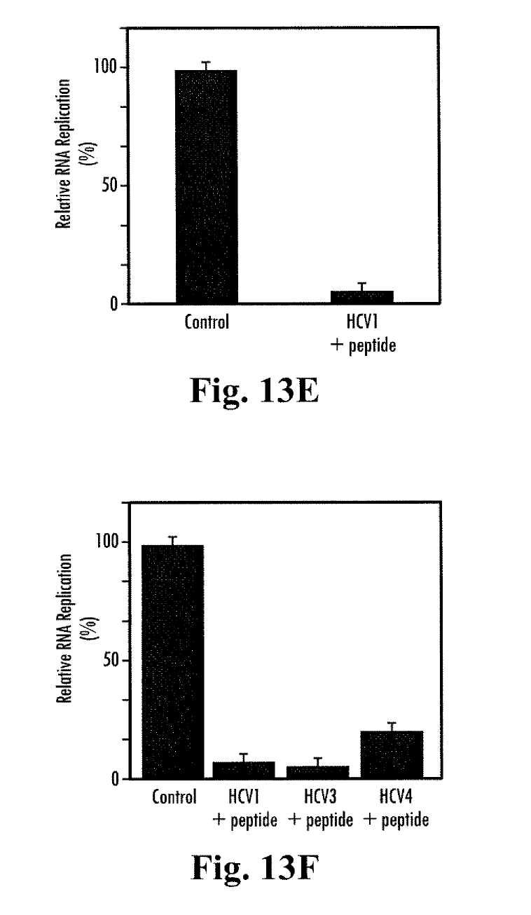

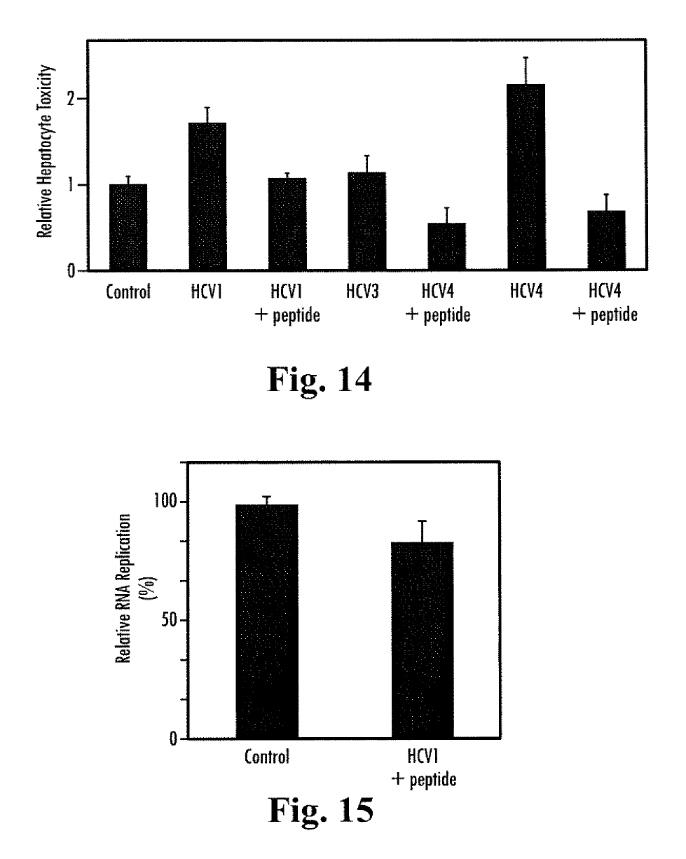

AP-50 peptide contains a 15-amino acid, cell permeable, leading sequence from the HIV-tat protein and FITC for fluorescent identification (FIG. 12). The AP-50 peptide prevented phosphorylation of endogenous AP-50 protein by recombinant HCV E2 in a cell-free kinase assay, with an IC₅₀ of ˜150 pM (FIG. 13A). As predicted, the AP-50 peptide was cell permeable as confirmed by the FITC fluorescence (FIG. 13B) and associated with HCV E2 in HCV-infected hepatocytes as determined by confocal microscopy (FIG. 13B). The HCV-infected liver as well as the HCV-infected human hepatocyte cultures displayed a marked increase in the phosphorylation of endogenous AP-50 on T¹⁵⁶, when compared to the uninfected liver or uninfected human hepatocyte culture (FIGS. 13C and 13D). Treatment of HCV-infected human hepatocyte cultures with the AP-50 peptide inhibited the phosphorylation of AP-50 on T¹⁵⁶ (FIG. 13D). Moreover, treatment of HCV-infected human hepatocyte cultures with the AP-50 peptide, inhibited HCV replication of genotypes 1, 3 and 4 at picomolar concentrations (FIGS. 13E and 13F), while improving the viability of HCV-infected hepatocytes (FIG. 14). The AP-50 peptide blocked HCV replication when given either at time zero of the infection (FIG. 13E), or after a 4 hr-infection (FIG. 13F). As expected, the phosphorylation mimic (QCE156VQ: SEQ ID:NO 1) AP-50 peptide linked to the HIV-tat and FITC, had no effect on HCV replication (FIG. 15).

What is claimed is:

1. An isolated peptide for inhibiting HCV infection comprising a peptide that inhibits one or more functional domains of HCV E2 protein from interacting with associated proteins selected from the group consisting of AP-50, HSC70, Cyclin A, and Cyclin G.

2. The isolated peptide of claim 1, wherein said peptide binds to an amino acid sequence as set forth in SEQ ID NO:1, SEQ ID NO:32, or SEQ ID NO:33, of HCV E2 protein.

3. The isolated peptide of claim 1, wherein said peptide is an AP-50 mutant.

4. The isolated peptide of claim 3, wherein said peptide comprises an AP-50 mutant comprising an amino acid sequence of SEQ ID NO:2 having an Alanine substitution at 156 position of a native AP-50.

5. The isolated peptide of claim 3, wherein said AP-50 mutant lacks a functional domain of a native AP-50.

6. The isolated peptide of claim 5, wherein said functional domain is a J domain of a native AP-50.

7. The isolated peptide of claim 1, wherein said peptide is a HCV E2 mutant.

8. The isolated peptide of claim t, wherein said peptide is a HSC70 mutant.

9. The isolated peptide of claim 1, wherein said peptide is a Cyclin G mutant.

10. The isolated peptide of claim 1, wherein said peptide is a Cyclin A mutant.

11. The isolated peptide of claim 1, wherein said peptide is an antibody.

12. The isolated peptide of claim 1, wherein said peptide is a vaccine.

13. A pharmaceutical composition for preventing or treating HCV infection comprising the isolated peptide of claim 1, and a pharmaceutically acceptable carrier.

14. The pharmaceutical composition of claim 13, wherein said peptide comprises an amino acid sequence of SEQ ID NO:2 having an Alanine substitution at position 156 of a native AP-50.

15. A method of preventing or treating HCV infection comprising administering to a subject in need an effective amount of an isolated peptide that inhibits one or more functional domains of HCV E2 protein from interacting with associated proteins selected from the group consisting of AP-50, HSC70, Cyclin A, and Cyclin G.

16. The method of claim 15, wherein said peptide binds to an amino acid sequence set forth in SEQ ID NO:1, SEQ ID NO:32, or SEQ ID NO:33, of HCV E2 protein.

17. The method of claim 15, wherein said peptide is an AP-50 mutant.

18. The method of claim 17, wherein said peptide comprises an AP-50 mutant comprising an amino acid sequence of SEQ ID NO:2 having an Alanine substitution at 156 position of a native AP-50.

19. The method of claim 17, wherein said AP-50 mutant lacks a functional domain of a native AP-50.

20. The method of claim 19, wherein said functional domain is a J domain of a native AP-50.

21. The method of claim 15, wherein said peptide is a HCV E2 mutant.

22. The method of claim 15, wherein said peptide is a HSC70 mutant.

23. The method of claim 15, wherein said peptide is a Cyclin G mutant.

24. The method of claim 15, wherein said peptide is a Cyclin A mutant.

25. The method of claim 15, wherein said peptide is an antibody.

26. The method of claim 15, wherein said peptide is a vaccine.

27. A primary hepatocyte cell culture comprising hepatocytes derived from a healthy subject and a bodily fluid derived from a HCV infected subject.

28. The primary hepatocyte cell culture of claim 27, wherein said bodily fluid is serum or plasma.

29. The primary hepatocyte cell culture of claim 27, wherein said bodily fluid is serum.

30. The primary hepatocyte cell culture of claim 27 comprising HCV genotypes 1, 2, 3, 4, or combinations thereof.

31. The primary hepatocyte cell culture of claim 27, wherein said subject is a human.

32. A method for screening a compound for inhibiting HCV infection, comprising
a) obtaining the primary hepatocyte cell culture of claim 22,
b) infecting said primary hepatocyte cell culture with HCV in the absence or presence of said compound, and
c) determining differences of HCV infection in the cultures in the absence or presence of said compound.


SEQUENCE LISTING (41 sequences)

SEQ ID NO: 1 - protein, 8 aa, Hepatitis C virus
  MOD_RES (3)..(4): Any amino acid
LIXXQXTG

SEQ ID NO: 2 - protein, 5 aa, Homo sapiens
QGAVQ

SEQ ID NO: 3 - DNA, 50 bp, Artificial Sequence
  Description of Artificial Sequence Synthetic primer
aatttaatacgactcactatagggacctcgcaagcaccctatcaggcagt

SEQ ID NO: 4 - DNA, 50 bp, Artificial Sequence
  Description of Artificial Sequence Synthetic primer
aatttaatacgactcactatagggacctcgcaagcgccctatcaggcagt

SEQ ID NO: 5 - DNA, 50 bp, Artificial Sequence; identical to SEQ ID NO: 3
  Description of Artificial Sequence Synthetic primer

SEQ ID NO: 6 - DNA, 24 bp, Artificial Sequence
  Description of Artificial Sequence Synthetic primer
gcagaaagcgtctagccatggcgt

SEQ ID NO: 7 - DNA, 50 bp, Artificial Sequence; identical to SEQ ID NO: 4
  Description of Artificial Sequence Synthetic primer

SEQ ID NO: 8 - DNA, 24 bp, Artificial Sequence
  Description of Artificial Sequence Synthetic primer
gcagaaagcgcctagccatggcgt

SEQ ID NO: 9 - DNA, 24 bp, Artificial Sequence
  Description of Artificial Sequence Synthetic primer
gcggaaagcgcctagccatggcgt

SEQ ID NO: 10 - DNA, 30 bp, Artificial Sequence
  Description of Artificial Sequence Synthetic primer
acaccaggcgccaggcagaacatccaactg

SEQ ID NO: 11 - DNA, 30 bp, Artificial Sequence
  Description of Artificial Sequence Synthetic primer
cagttggatgttctgcctggcgcctggtgt

SEQ ID NO: 12 - DNA, 30 bp, Artificial Sequence
  Description of Artificial Sequence Synthetic primer
ggcaacaacaccttggcatgccccactgat

SEQ ID NO: 13 - DNA, 30 bp, Artificial Sequence
  Description of Artificial Sequence Synthetic primer
atcagtggggcatgccaaggtgttgttgcc

SEQ ID NO: 14 - DNA, 30 bp, Artificial Sequence
  Description of Artificial Sequence Synthetic primer
tgcatggtcgacgagccgtataggctttgg

SEQ ID NO: 15 - DNA, 30 bp, Artificial Sequence
  Description of Artificial Sequence Synthetic primer
ccaaagcctatacggctcgtcgaccatgca

SEQ ID NO: 16 - DNA, 30 bp, Artificial Sequence
  Description of Artificial Sequence Synthetic primer
tgcatggtcgacttcccgtataggctttgg

SEQ ID NO: 17 - DNA, 29 bp, Artificial Sequence
  Description of Artificial Sequence Synthetic primer
caaagcctatacgggaagtcgaccatgca

SEQ ID NO: 18 - DNA, 30 bp, Artificial Sequence
  Description of Artificial Sequence Synthetic primer
cgctgtgatctggctgacagggacaggtcc

SEQ ID NO: 19 - DNA, 30 bp, Artificial Sequence
  Description of Artificial Sequence Synthetic primer
ggacctgtccctgtcagccagatcacagcg

SEQ ID NO: 20 - DNA, 30 bp, Artificial Sequence
  Description of Artificial Sequence Synthetic primer
ctggaagacagggccaggtccgagctcagc

SEQ ID NO: 21 - DNA, 30 bp, Artificial Sequence
  Description of Artificial Sequence Synthetic primer
gctgagctcggacctggccctgtcttccag

SEQ ID NO: 22 - DNA, 30 bp, Artificial Sequence
  Description of Artificial Sequence Synthetic primer
ctcagcccgttagcactgaccactacacag

SEQ ID NO: 23 - DNA, 30 bp, Artificial Sequence
  Description of Artificial Sequence Synthetic primer
ctgtgtagtggtcagtgctaacgggctgag

SEQ ID NO: 24 - DNA, 30 bp, Artificial Sequence
  Description of Artificial Sequence Synthetic primer
acacagtggcaggtcgcaccgtgttccttc

SEQ ID NO: 25 - DNA, 30 bp, Artificial Sequence
  Description of Artificial Sequence Synthetic primer
gaaggaacacggtgcgacctgccactgtgt

SEQ ID NO: 26 - DNA, 30 bp, Artificial Sequence
  Description of Artificial Sequence Synthetic primer
cacctccaccagaacgcagtggacgtgcag

SEQ ID NO: 27 - DNA, 30 bp, Artificial Sequence
  Description of Artificial Sequence Synthetic primer
ctgcacctccactgcgttctggtggaggtg

SEQ ID NO: 28 - DNA, 30 bp, Artificial Sequence
  Description of Artificial Sequence Synthetic primer
gcgtcctgggccgcaaagtgggagtacgtc

SEQ ID NO: 29 - DNA, 30 bp, Artificial Sequence
  Description of Artificial Sequence Synthetic primer
gacgtactcccactttgcggcccacgacgc

SEQ ID NO: 30 - DNA, 30 bp, Artificial Sequence
  Description of Artificial Sequence Synthetic primer
gttctcctgttccttgcacttgcagacgcg

SEQ ID NO: 31 - DNA, 30 bp, Artificial Sequence
  Description of Artificial Sequence Synthetic primer
cgcgtctgcaagtgcaaggaacaggagaac

SEQ ID NO: 32 - protein, 9 aa, Hepatitis C virus
SGREYALKR

SEQ ID NO: 33 - protein, 16 aa, Hepatitis C virus
LVGLLTPGAKQNIQLI

SEQ ID NO: 34 - protein, 5 aa, Homo sapiens
QCEVQ

SEQ ID NO: 35 - protein, 15 aa, Artificial Sequence
  Description of Artificial Sequence Synthetic peptide
XXHXDXKPQNXLMLX

SEQ ID NO: 36 - protein, 14 aa, Hepatitis C virus
LLTPGAKQNIQLIN

SEQ ID NO: 37 - protein, 14 aa, Hepatitis C virus
LLTPGARQNIQLIN

SEQ ID NO: 38 - protein, 201 aa, Homo sapiens
MSLLQSALDFLAGPGSLGGASGRDQSDFVGQTVELGELRLRVRRVLAEGGFAFVYEAQDVGSGREYALKRLLSNEEEKNRAIIQEVCFMKKLSGHPNIVQFCSAASIGKEESDTGQAEFLLLTELCKGQLVEFLKKMESRGPLSCDTVLKIFYQTCRAVQHMHRQKPPIIHRDLKVENLLLSNQGTIKLCDFGSATTISHY

SEQ ID NO: 39 - protein, 201 aa, Hepatitis C virus
LGNWFGCTWMNSTGFTKVCGAPPCVIGGVGNNTLLCPTDCFRKHPEATYSRCGSGPWITPYCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 40 - protein, 201 aa, Hepatitis C virus
  MOD_RES (66)..(66): Phe or Ala
LGNWFGCTWMNSTGFTKVCGAPPCVIGGVGNNTLACPTDCFRKHPEATYSRCGSGPWITPYCMVDXPYRLWHYPCTINYTIFKVRMYVGGVEHRLEAACNWTRGERCDLADRARSELSPLALSTTQWQVAPCSFTTLPALSTGLIHLHQNAVDVQYLYGVGSSIASWAAKWEYVVLLFLALADARVCSCLWMMLLISQAEA

SEQ ID NO: 41 - protein, 20 aa, Artificial Sequence
  Description of Artificial Sequence Synthetic peptide
GGGGYGRKKRRQRRRQVAGQ